(19) United States
(12) Patent Application Publication
(10) Pub. No.: US 2025/0308641 A1
(43) Pub. Date: Oct. 2, 2025

(54) Hybrid Molecule-Based (MB) Attribute Model (AM) Framework for Generic Aggregated Molecular Mixtures of Sustainable Feedstocks

(71) Applicant: AspenTech Corporation, Bedford, MA (US)

(72) Inventors: Zhen Hou, Lexington, MA (US); Darin Campbell, Waltham, MA (US)

(21) Appl. No.: 18/621,620

(22) Filed: Mar. 29, 2024

(51) Int. Cl. G16C 20/30; G16C 20/10

(52) U.S. Cl. CPC G16C 20/30; G16C 20/10

(57) ABSTRACT

Embodiments represent a composition of molecules, in a chemical feedstock as a probabilistic combination of (i) a subset of a plurality of structural attribute representations and (ii) a subset of a plurality of individual molecule representations. Then, representations of reaction paths and reaction kinetics are determined based on the plurality of structural attribute representations and the plurality of individual molecule representations. A simulation is automatically performed of a chemical process on the feedstock that results in a processed feedstock using (i) the subset of the plurality of structural attribute representations, (ii) the subset of the plurality of individual molecule representations, and (iii) the determined representations of the reaction paths and reaction kinetics. As a result of the simulation, properties of the processed feedstock are automatically determined by a computer-based system, and improved modeling of feedstock in chemical processes is achieved as applied to process control, monitoring, maintenance, scheduling, etc.

### BACKGROUND

Existing computer-based methods and systems for modeling chemical reactions can model thousands of species and similarly, thousands of reactions. However, these existing methods are not capable of modeling structures and reactions in certain circumstances, such as when modeling reactions involving sustainable feedstocks.

Accordingly, there is a need for improved computer-implemented methods and systems for modeling chemical reactions.

### SUMMARY

Embodiments of the present invention provide methods and systems for modeling chemical feedstocks in a chemical process. Amongst other examples, embodiments can model chemical feedstocks to determine the properties of the feedstock. The determined properties may be physical, structural, physics-based, and/or chemistry-based and the like.

One such example embodiment is directed to a method for determining properties of a chemical feedstock. In an embodiment, the method represents, in computer memory, composition of molecules (that includes any of an amorphous solid and a polymeric material) in a chemical feedstock as a probabilistic combination of (i) a subset of a plurality of structural attribute representations, wherein the plurality of structural attribute representations includes cores, inter-core linkages, and free-terminal inter-core linkages, and (ii) a subset of a plurality of individual molecule representations. The method continues and determines, in the computer memory, representations of reaction paths and reaction kinetics in terms of the plurality of structural attribute representations and the plurality of individual molecule representations. Next, such an embodiment performs a simulation of a chemical process on the chemical feedstock that results in a processed feedstock. The simulation is performed using the subset of the plurality of structural attribute representations, the subset of the plurality of individual molecule representations, and the determined representations of the reaction paths and reaction kinetics. In turn, a property of the processed feedstock is determined based on results of performing the simulation.

In embodiments, the steps of the method, e.g., the receiving, formulating, simulating, and sampling may be automatically performed or may be performed responsive to user input.

According to an embodiment, a given free-terminal inter-core linkage represents a side chain. An embodiment also receives, in computer memory, an indication of at least one of: the subset of the plurality of structural attribute representations and the subset of the plurality of individual molecule representations.

According to an embodiment, the chemical process is a chemical reaction. In another embodiment, the chemical reaction is a combination reaction, a decomposition reaction, a single-replacement reaction, a double-replacement reaction, or a reactor, e.g., refinery reactor, reaction. In yet another embodiment, said reactor reaction comprises: polymerization, depolymerization, saturation, desaturation, hydrodesulfurization (HDS), hydrodenitrogenation (HDN), ring-opening, ring-closing, addition reaction, elimination reaction, hydrodemetallization (HDM), hydrodeoxygenation (HDO), carbonylation, decarbonylation, carboxylation, decarboxylation, hydrolysis, hydroisomerization, acid cracking, metal cracking, ring condensation, or ring expansion. Further, in an embodiment, a reactor reaction may be defined as a chemical reaction that is performed in a chemical reactor for the generation of desired products (e.g., upgrading a petroleum feedstock).

According to an embodiment, the chemical process is a separation. In another embodiment, the separation is at least one of: a vapor-liquid equilibrium separation, a liquid-liquid equilibrium separation, and a solid-liquid equilibrium separation.

An embodiment performs the simulation of the chemical process using user input. For instance, an example embodiment of a user input is at least one of: a physical property, a thermodynamic property, or one or more chemical structures of an attribute of the chemical feedstock.

An example embodiment of a chemical feedstock is at least one of: petroleum resid, lignin, cellulose, and a plastic. According to yet another example embodiment, the property determined for the processed feedstock is at least one of: a thermodynamic property, a physical property, and a molecular distribution. With the determined property of the subject feedstock, embodiments provide improved chemical feedstock models in a given chemical process. The improved modeling of chemical feedstocks in chemical processes in turn provides advantages in process control, monitoring, maintenance, and scheduling. For example, results from simulations performed using the improved modeling can be used to take real-world actions to control the real-world chemical process. In an embodiment, the model may be used for reactor performance monitoring, real time online optimization, or maintenance and scheduling. In reactor performance monitoring, a user may see the reactor and plant performance and make additional decisions such as feed selection and changes to operating conditions to maximize profit and minimize harmful emissions. In a non-limiting example, operating conditions may include temperature, pressure, feed rate, etc. In real time online optimization, the model may make decisions on key operating parameters and set the parameters directly to optimize an objective function. In maintenance and scheduling, the model can predict how model performance is degrading due to indicators, such as coking, deactivation of catalyst, or contamination. The user can then make decisions on when to shut down or how to modify the operation of a unit to extend the time until shutdown is required.

Another embodiment is directed to a system for determining properties of a chemical feedstock. The system includes a processor and a memory with computer code instructions stored thereon. The processor and the memory, with the computer code instructions, are configured to cause the system to implement any embodiments or combination of embodiments described herein.

Yet another embodiment is directed to a computer program product for determining properties of a chemical feedstock. The computer program product comprises a computer readable medium with computer code instructions stored thereon where, the computer code instructions, when executed by a processor, cause an apparatus associated with the processor to perform any embodiments or combination of embodiments described herein.

It is noted that embodiments of the method, system, and computer program product may be configured to implement any embodiments, or combination of embodiments, described herein.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-E are chemical or molecular depictions of representative feedstocks used in sustainable chemical conversions.

FIG. 2 is a schematic illustration of a sampling methodology that may be utilized in embodiments.

FIG. 3 is a flowchart depicting a method for determining properties of a chemical feedstock according to an embodiment.

FIG. 4 is a representation of a complex molecule that may be analyzed using embodiments.

FIG. 5 is a schematic depiction of a combination of complex molecules that may be described using embodiments.

FIG. 6 depicts chemical or other reactions, or the like, that may be simulated by embodiments.

FIG. 7 depicts molecule representations that may be implemented and employed in embodiments.

FIGS. 8-9 are schematic depictions of chemical or other reactions, or the like, that can be simulated by embodiments.

FIGS. 10-13 depict molecule representations that may be implemented and employed in embodiments.

FIGS. 14-16 are schematic depictions of chemical or other reactions, or the like, that may be simulated using embodiments.

FIGS. 17-18 illustrate representations of molecules that may be implemented and employed in embodiments.

FIG. 19 is a schematic depiction of chemical or other reactions, or the like, that can be simulated by embodiments.

FIG. 20 depicts molecule representations that may be implemented and employed in embodiments.

FIG. 21 is a flowchart of an embodiment for simulating a chemical reaction to determine products of the reaction.

FIG. 22 is a schematic view of a computer network or similar digital processing environment in which embodiments of the present invention may be implemented.

FIG. 23 is a block diagram of an example internal structure of a computer in the environment of FIG. 22.

### DETAILED DESCRIPTION

A description of example embodiments follows.

The teachings of all patents, published applications, and references cited herein are incorporated by reference in their entirety.

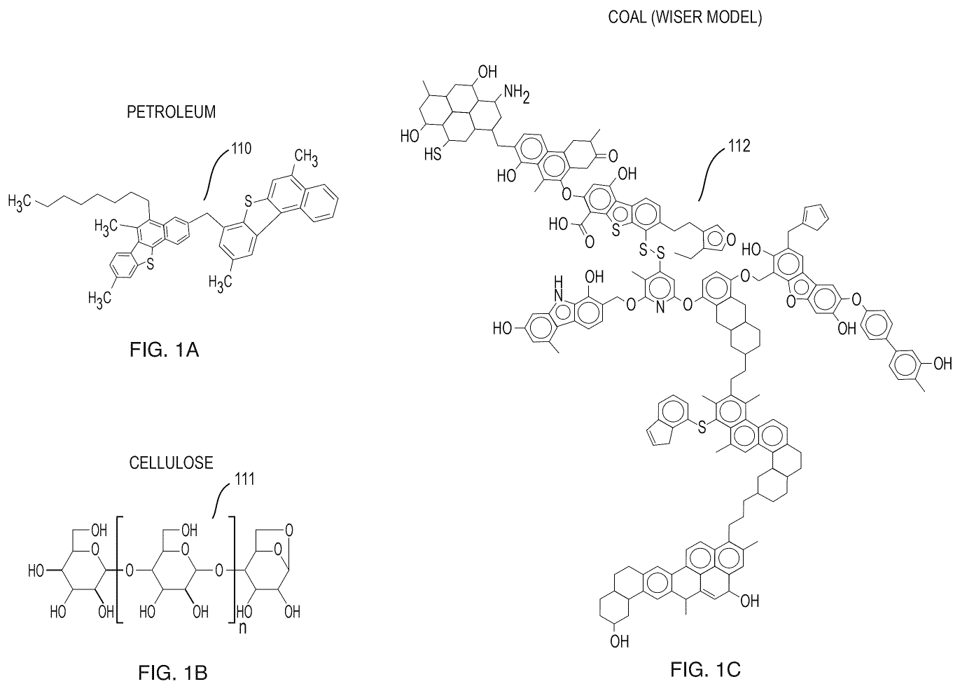

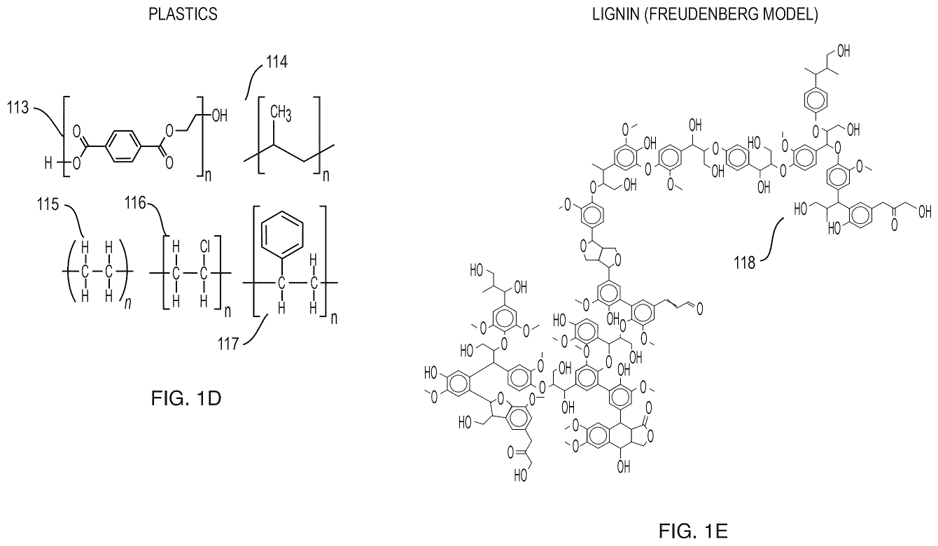

Demand for sustainable energy around the world is increasing, and energy and fuel industries are upgrading a variety of alternative feedstocks in addition to petroleum. FIGS. 1A-E show a variety of feedstocks 110 (petroleum), 111 (cellulose), 112 (coal), 113-117 (plastics), and 118 lignin, used in sustainable chemical conversions, indicating high valued, fuel and material-derived products.

Petroleum, 110, oils illustrated in FIG. 1A are still a major source for energy and materials; however, they can be a part of sustainable chemical processes to pursue high valued products while satisfying the standard of carbon emission by use of techniques such as carbon capture and sequestration (CCUS). Improving understanding of heavy resid or asphaltene is a challenge in petroleum refining to optimize upgrading sustainable chemical processes and reducing carbon emissions, which is challenging due to the complexity of the structures and chemistries. There are hundreds of distinct aggregated ring structures in a fraction of petroleum heavy resid that determine the reactivity, thermodynamics, and key properties of petroleum. Modeling a large number of complex structures and their related chemistries is a challenge for current modeling capabilities.

Circular economics are important to sustainable chemical conversions. Upgrading waste plastics to valuable products is a major route. For example, plastic pyrolysis oils serve as alternative feedstocks for the refining industry; however, it is challenging to describe the structures of plastics, chemistries of plastics conversions, and components of products derived from plastics. The structures of plastics (e.g., the structures 113-117 shown in FIG. 1D) are indefinitely long polymers with enormous numbers of intermediates and products generated during the upgrading process, making it challenging to keep all important details using current deterministic modeling methodology.

Another source of sustainable feedstocks are wood wastes. Cellulose/hemi-cellulose 111 and lignin 118 are major sources of wood wastes. Like plastics, wood wastes are highly aggregated and polymerized complex mixtures as shown by the structure representations 111 and 118 of FIGS. 1B and 1E, respectively. Current modeling techniques are unable to illustrate the chemistries of wood wastes upgrading as their molecular details need to be described, but modeling large numbers of aggregated complex mixtures is limiting.

Moreover, certain regions of the world are still showing high interest in coal. The structures of coal (e.g., 112 illustrated in FIG. 1C) are more complex than lignin, cellulose, and plastics. Coals consists of highly aggregated and polymerized structures, and a variety of different heteroatom containing moieties. As a result, it is challenging to describe a structure and derived chemical processes from coal using current modeling techniques.

Aspen Technology Inc. (Assignee) previously invented and disclosed a Hybrid Attribute Reaction Model (ARM) in Molecule-Based EO (Equation Oriented) Reactor (MB EORXR) (U.S. patent application Ser. No. 16/739,291 published as U.S. Patent Publication 2021/0217497 A1, hereinafter '291 Application) that could effectively reduce the computational resources for a complex petroleum heavy oil system. Using the ARM MB EORXR method, complex molecules can be defined by two attributes: molecular type (MT) and main side chains (main SC). The methods described in the '291 application are applicable to systems that have a limited number of individual molecular types: O (100)˜O (2000) (where “O” indicates “on the order of”). The molecular structures the methods of the '291 application can describe are “island” molecules and “archipelago” molecules that have a limited number of moieties which could be explicitly juxtaposed manually as “super large island” structures.

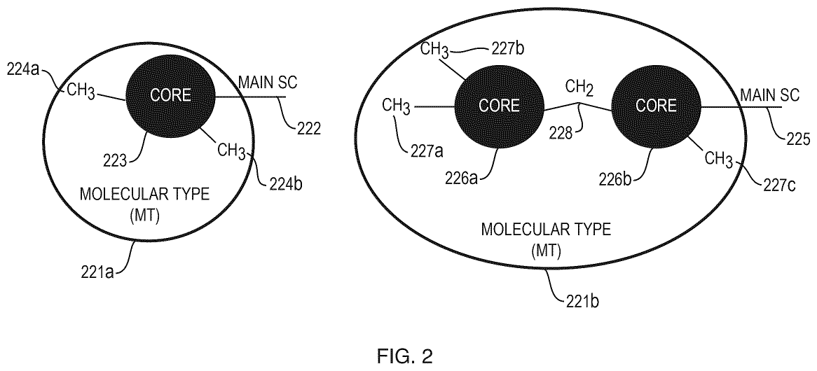

FIG. 2 illustrates an example methodology for representing complex molecules in the ARM MB EORXR method according to an embodiment. In particular, the FIG. 2 illustrates a molecular type representation 221a, along with a juxtaposed “super large island” structure 221b. The molecular representation 221a comprises a core 223, which has a main side chain 222 bound to the core 223, along with two other side chains 224a and 224b that are shown as methyl groups (—CH₃) in the depiction 221a. The super large island structure 221b depicts how complex molecules are bound together according to an embodiment. The super large island structure representation 221b includes two cores 226a and 226b bound together and connected by a carbon linker 228 (—CH₂—). Both cores 226a-b have side chains bound to each of them 227a-b (bound to core 226a) and 227c (bound to core 226b), with each depicted as methyl groups (—CH₃). Additionally, the main side chain 225 is bound to the core 226b. Despite the significant improvements provided by using the representation functionality depicted in FIG. 2, the ARM MB EORXR method is unable to describe aggregated complex structures with highly polymerized moieties such as plastics, cellulose, lignin, coal, and other complex archipelago structures.

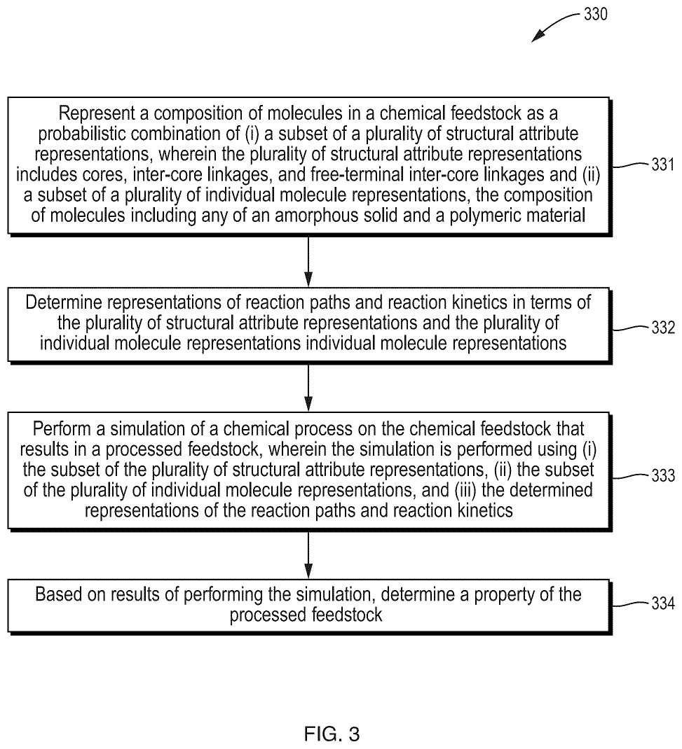

Given limitations of property estimations in existing methods, e.g., the '291 application, a new approach is needed to determine properties of chemical feedstocks to describe core size and quantify how products differ from chemical feedstocks. An example embodiment expands the MB ARM framework to incorporate new properties for complex structures. In particular, an embodiment introduces the Flory p parameter to enable calculating properties describing core size and quantify how much the products differ from the feedstock. FIG. 3 illustrates an example method 330 for determining properties of feedstocks. The method 330 is computer-implemented and may be performed via any combination of hardware and software as is known in the art. For example, the method 330 may be implemented via one or more processors with associated memory storing computer code that causes the processor to implement steps 331, 332, 333, and 334 of the method 330. Further, the method 330 may be implemented in existing simulation software such as Aspen Technology, Inc.'s (Assignee's) Hybrid Attribute Reaction Model (ARM) in Molecule-Based EO (Equation Oriented) Reactor (MB EORXR) described in U.S. patent application Ser. No. 16/739,291. In such an implementation, the method 330 and/or any other embodiments described herein, may be implemented in the ARM MB EORXR block. Further, it is noted that herein embodiments are described as being capable of being implemented in existing software products or systems supported by Applicant, however, embodiments are not limited to being implemented into existing software and, instead, embodiments can be performed using any combination of hardware and software as is known in the art. Embodiments may be part of the software system or suite that supports, monitors, controls, provides maintenance of, etc. industrial processing plants, refineries, chemical or pharmaceutical processing plants, and the like.

Returning to FIG. 3, the method 330 begins at step 331 by representing, e.g., in computer memory, a composition of molecules in a chemical feedstock as a probabilistic combination of representations, namely, a subset of a plurality of structural attribute representations and a subset of a plurality of individual molecule representations. According to an embodiment of the method 330, the structural attribute representations include cores, inter-core linkages, and free-terminal inter-core linkages. According to an embodiment, cores represent complex molecular pieces of a large number of aggregated ring structures and multiple different structural functional groups. Moreover, inter-core linkages are generally linear structures between cores and free-terminal inter-core linkages are structures that are attached to only one core.

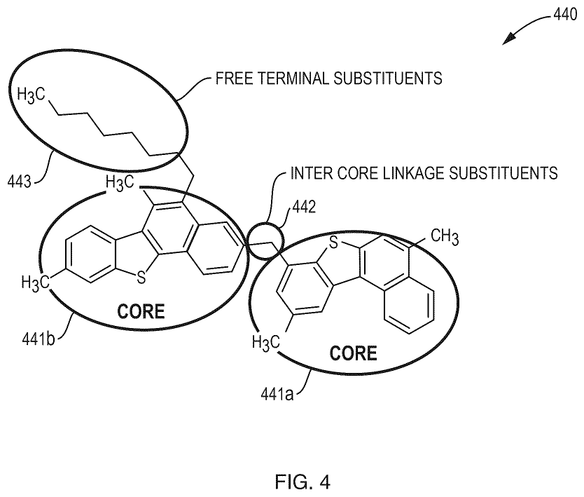

In an embodiment of the method 330, the composition of molecules that is represented at step 331 includes any of amorphous solids and polymeric material. Further examples of compositions of molecules that can be represented at step 331 are described hereinbelow in relation to FIG. 4. Further, it is noted that the method 330 may be used to simulate a chemical feedstock known to those of skill in the art. For example, in embodiments the chemical feedstock may be at least one of: petroleum resid, coal, lignin, cellulose, and a plastic.

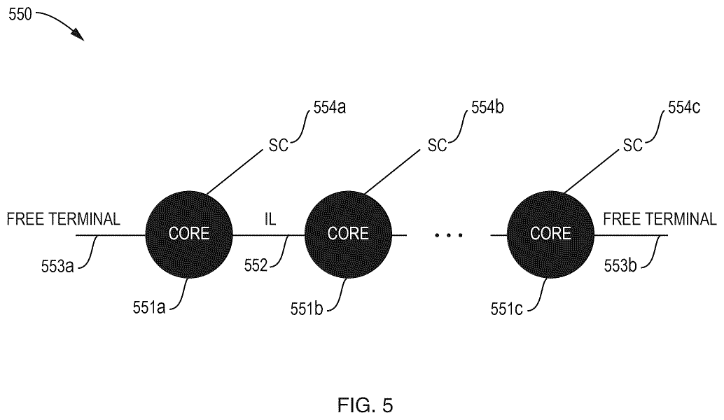

Step 331 of FIG. 3 may use the abstraction functionality described hereinbelow in relation to FIG. 5 to represent the composition of molecules. Example structural attribute representations and individual molecule representations that may be utilized in embodiments of the method 330 are described hereinbelow in relation to FIG. 5. It is noted that in an embodiment of the method 330, there are a plurality of structural attribute representations and a plurality of individual molecule representations that may be utilized at step 331 to represent the composition of molecules.

From the plurality of structural attribute representations and the plurality of individual molecule representations that may be utilized, particular subsets are used to represent the composition of molecules of interest at step 331. In other words, there are a plurality of structural attribute representations and a plurality of individual molecule representations and, according to an embodiment, some subset, e.g., portion, of each of the plurality of structural attribute representations and the plurality of individual molecule representations are used at step 331 to represent the composition. In another embodiment of the method 330, a linear polymer-like structure defines a portion of the composition of molecules of interest at step 331 (which may be a complex polymerized molecule containing multiple cores, inter-core linkages, and free-terminal linkages). In such an embodiment (i.e., where a linear polymer-like structure defines a portion of the composition of molecules), the polymerized molecule can be further represented in the method 330 based on the attributes including the cores, inter-core linkages, and potential free-terminal linkages. Further, in an embodiment, cores may contain special free-terminal linkages called side chains that go beyond the definition of linear polymer statistics, creating different characteristics for different chemical feedstocks. In another embodiment, an amorphous solid is comprised of polymerized complex molecules and individual components of polymerized complex molecules, such as individual cores with or without free-terminal linkages.

To continue, the method 330 continues at step 332 by determining, in computer memory, representations of reaction paths and reaction kinetics in terms of the plurality of structural attribute representations and the plurality of individual molecule representations. To further illustrate, a particular subset of the plurality of structural attribute representations, and a particular subset of the plurality of individual molecule representations, are utilized at step 331 to represent the composition of molecules but, at step 332, the determined representations of the reaction paths and the reaction kinetics are determined in terms of the entire plurality of structural attribute representations and the entire plurality of individual molecule representations. According to an embodiment, the reaction paths may comprise a combination reaction, a decomposition reaction, a single-replacement reaction, a double-replacement reaction, or a refinery reactor reaction. More specifically, the reactions may comprise: polymerization, depolymerization, saturation, desaturation, hydrodesulfurization (HDS), hydrodenitrogenation (HDN), ring-opening, ring-closing, addition reaction, elimination reaction, hydrodemetallization (HDM), hydrodeoxygenation (HDO), carbonylation, decarbonylation, carboxylation, decarboxylation, hydrolysis, hydroisomerization, acid cracking, metal cracking, ring condensation, or ring expansion. In another embodiment, the reaction kinetics may comprise of at least one of: a thermodynamic property, a physical property, or one or more chemical structures of an attribute of the chemical feedstock. In such embodiments, the reaction paths and reaction kinetics can be based on user input. According to an embodiment, a user specifies desired reactions and rate laws for the MB ARM model and, in turn, the model converts reaction paths and reaction kinetics to code that includes equations for the reactions and reactor type for possible reactions permissible with permissible components. In an embodiment, the composition can be directly specified from the feed stream to the reactor or additional data, e.g., distillation, gravity, paraffins, and olefins, naphthenes, and aromatics (PONA), amongst other examples, may be incorporated into the model to further calculate the composition of the feedstock from the reaction paths and reaction kinetics input.

At step 333 the method 330 continues by performing a simulation of a chemical process on a chemical feedstock that results in a processed feedstock. The simulation is performed at step 333 using (i) the subset of the plurality of structural attribute representations of step 331, (ii) the subset of the plurality of individual molecule representations of step 331, and (iii) the representations of the reaction paths and reaction kinetics determined by and output from step 332 that simulates structural attribute representations, individual molecule representations, and representations of reaction paths and reaction kinetics from a user input. In addition to using (i) the subset of the plurality of structural attribute representations of step 331, (ii) the subset of the plurality of individual molecule representations of step 331, and (iii) the determined representations of the reaction paths and reaction kinetics output from step 332, the simulation may also be performed at step 333 utilizing user input. Example user input that may be utilized includes indications of at least one of: a physical property (e.g., melting point, boiling point, or density), a thermodynamic property (e.g., temperature or pressure), or one or more chemical structures of an attribute of the chemical feedstock, for a non-limiting example. In another embodiment, in addition to performing the simulation at step 333 using (i) the subset of the plurality of structural attribute representations of step 331, (ii) the subset of the plurality of individual molecule representations of step 331, and (iii) the determined representations of the reaction paths and reaction kinetics output from step 332, an embodiment of the method 330 may also determine temperature and pressure from a feed stream, as well as any other relevant information determined from the reactor, such as diameter, catalyst loading, and void fraction and evaluate equations based on values of EO variables. Embodiments, at step 333, can iteratively calculate the specified variables until each residual equation satisfies a specified tolerance, e.g., is close to zero. In such an embodiment, when equations in the model are converged, the calculations of the simulation at step 333 are complete, and the results of the simulation, such as compositions, properties, and reactor relevant information, can be populated by the model. In an embodiment, the model may populate variables as an outlet of the reactor, and the model can populate the composition, properties, and relevant reactor information into variables that are associated with the outlet of the reactor.

In an embodiment, the chemical process that is simulated at step 333 comprises a chemical reaction. In another embodiment, a chemical process comprises a separation. In one such embodiment, the separation comprises at least one of: a vapor-liquid equilibrium separation, a liquid-liquid equilibrium separation, and a solid-liquid equilibrium separation.

To continue, at step 334, properties of the processed feedstock are determined based on results of the simulation (output of 333). In an embodiment, a property of the processed feedstock that is determined at step 334 is at least one of: a thermodynamic property, a physical property, and a molecular distribution. An embodiment provides a new hybrid attribute modeling framework (AMF) approach. One such AMF framework may be implemented in the MB (Molecular-Base) system available from Aspen Technology, Inc (Assignee). Other molecular-based systems may be suitable. The hybrid molecular attribute-based modeling framework described herein can describe a large scale of process systems with a variety of sustainable feedstocks containing highly polymerized complex moieties with complicated multi-functional structures while maintaining full molecular details and robust convergence performance. In a non-limiting example embodiment, for some properties, e.g., sulfur content, the process is a blending of mass, volume, or mole basis. In such an example embodiment, the properties of the feedstock are determined at step 334 based on results of the simulation by taking the mass, volume or molar composition of molecules or attributes and multiplying by a property value and summing to determine the property value of the mixture. Other properties have nonlinear blending rules (e.g., research octane number). In such an example embodiment, the property value and composition of each molecule or attribute are used as inputs into a blending equation to determine the blended property of the mixture.

With the determined property of the subject feedstock, embodiments provide improved chemical feedstock models of a given chemical process. The improved modeling of chemical feedstock in chemical processes in turn provides advantages in process control, monitoring, maintenance, and scheduling. In a non-limiting example embodiment, advantages observed in process control, monitoring, maintenance, and scheduling benefit factory operations such as determining separations necessary for a reaction and determining reactor conditions for complex molecules that were previously challenging to perform computationally, while using an affordable computational time and maintain full molecular details. Further, monitoring and maintenance are beneficial for overall safety and cost reduction in non-limiting embodiments. Embodiments can be used to determine properties of feedstocks that could previously not be determined. Further, the results from embodiments can be used to control and modify real-world processes. For instance, an embodiment may determine the property of a feedstock subject to a manufacturing process and, in response to the determined property, modify an aspect of the manufacturing process to improve results. In other non-limiting embodiments, the model can be used to characterize molecular details of aggregated new feedstocks such as lignin, cellulose, coal, or plastics. In terms of molecular components, non-limiting embodiments include obtaining pyrolysis oils from feedstocks that are used to model, simulate, and optimize/control upgrading processes of the feedstocks. In other non-limiting embodiments, those processes include hydro processing, catalytic cracking, etc. In other non-limiting embodiments, feedstocks can be represented/modelled which, with existing methods would have required almost infinite sets of molecules to represent the detailed information for characterizations and processes. As such, embodiments provide modelling which has not been achieved by previous methods known in the art.

An embodiment deconstructs a highly aggregated molecule to three essential attribute types (cores, side chains, and inter-core linkages). A statistical MB sampling protocol is utilized in an embodiment to define highly polymerized complex components in Aspen Technology, Inc.'s (Assignee's) MB Framework and considers isomers of small molecules.

In a non-limiting example embodiment, reactions and kinetics of complex chemistries are modeled by being written in terms of a limited number of attributes for highly aggregated complex mixtures. The reactions and kinetics of lighter fractions derived from those complex structures can be represented by individual components. The combination of structural attributes and individual components can be solved together to describe an upgrading process of highly polymerized complex components. The full molecular details including both structural attributes and individual molecules are maintained during the reactor simulation.

Further, in embodiments, the MB sampling protocol enables users to reversibly sample highly polymerized complex mixtures as a set of discretely sampled components from the collection of structural attributes and further map the highly polymerized complex mixtures into selected individual molecules. Implementing embodiments, e.g., AMF model (WO 2023/193172 A1), in Aspen Technology, Inc.'s (Assignee's) molecule-based rigorous reversible lumping (MB RRL) method to highly aggregated molecules, enables mapping of the highly aggregated complex mixtures to a set of thermodynamic lumps for various separation models and can be used to estimate curve-based properties.

In an embodiment, Aspen Technology, Inc.'s (Assignee's) MB Framework adds additional functionality to provide unit operations and property estimations in addition to reactors for highly polymerized complex mixtures including at least: mixer, splitter, flash, and property estimation.

In an example embodiment, Aspen Technology, Inc.'s (Assignee's) MB Framework and model builder are enhanced to support automation of code generation for new hybrid attribute modeling frameworks including at least residuals, sparsity, and jacobians in terms of Aspen Technology, Inc.'s (Assignee's) EO format.

Further, in embodiments., the AMF model, saves computational time while maintaining the full molecular details of a complex process system of a variety of highly polymerized structures. As a result, embodiments provide fast and robust performance to solve a complex flowsheet for sustainable feedstocks.

Implementing embodiments, e.g., the AMF model, allows users to create molecular level process models for sustainable feedstocks. Attribute types and a new MB sampling protocol are used to describe highly polymerized complex components including at least plastics, cellulose, lignin, and coal. Individual components can be reversibly sampled to/from collections of attributes and work together with the collection of attributes. Reactors and other separation process unit operations in the MB framework are updated to support the embodiments described herein. As a result, embodiments, e.g., the hybrid attribute modeling framework, can model complex sustainable feedstocks (such as the feedstocks 110-118 as shown in FIGS. 1A-E) in a complex flowsheet with affordable computational time while maintaining full molecular details. According to an embodiment, a complex flowsheet is a flowsheet with many pieces of equipment and/or a piece of equipment that has many equations in just one model. For example, a hydrocracker by itself would be considered a complex flowsheet as it may contain up to 12 reactor beds with mixers for quenches and separators for separating recycle gas from liquid products. A flowsheet may also include but is not limited to, fractionation, reactor units, flow splitters, heaters, heat exchangers, and extractive distillation units.

A general complex molecule 440 that embodiments, e.g., method 330, can determine properties of is shown in FIG. 4. The molecule 440 can be described by three essential structural attributes: core 441a-b, free-terminal substituents (FTIL) 443, and inter-core linkage substituents (IL) 442. Core, 441a-b, represents key complex molecular pieces including a large number of aggregated ring structures and multiple different structural functional groups. For example, hundreds of such complex molecular types exist in a heavy oil resid fraction and coals, as described in U.S. Pat. No. 11,101,020B2; Zhang, Linzhou, et al. “Molecular representation of petroleum vacuum resid” Energy & Fuels 28.3 (2014): 1736-1749; Zhang, Yunlong. “Identify Similarities in Diverse Polycyclic Aromatic Hydrocarbons of Asphaltenes and Heavy Oils Revealed by Noncontact Atomic Force Microscopy: Aromaticity, Bonding, and Implications in Reactivity” (2019). Extending to other polymerized materials: lignin, cellulose, and plastics, cores are used to describe a distinct repeat unit in the aggregated molecule. For example, a single aromatic ring with well-defined methyl, methoxy, and alcohol groups can be defined as a core for lignin. The bonding site of two monomers can be defined as a core for plastics and cellulose. Inter-core linkage substituents 442, in general, are linear structures between cores. There are O (10˜20) inter core linkage substituents in heavy resid, coal, and lignin. Extending to other polymerized materials: cellulose and plastics, inter-core linkage substituents 442 are distinct repeat units other than cores. On the other hand, free-terminal substituents, 443, are structures attached to one core only.

Because, as described above, embodiments implement and utilize a new paradigm where complex compositions of molecules can be described using cores, inter-core linkages, and free-terminal inter-core linkages, embodiments can abstract a general complex molecule 550 as shown in FIG. 5. Bethe lattice statistics is a well-known methodology that has been used to describe highly polymerized complex structures for years. To simplify the details for engineering usage, an embodiment can reduce Bethe statistics to a modified linear polymer statistic to represent both linear polymerized structures and cross-linked structures.

FIG. 5 depicts an abstraction of a polymerized complex molecule 550. FIG. 5 illustrates an embodiment where a linear polymer-like structure 550 is used to define the major portion of a complex polymerized molecule. In the structure 550, each core 551a-c has two binding sites to link with either IL 552, FTIL 553a-b, or a side chain (SC) 554a-c. Each IL 552 has two binding sites to link with two cores, e.g., the IL 552 links to cores 551a-b. There are two FTILs 553a-b shown at the head and the tail of core 551a and core 551c in FIG. 5. In addition to a typical linear polymer structure, embodiments may have different branched moieties with cores and ILs that define a set of various distinct structures. By explicitly illustrating branched structural units in cores and ILs, embodiments can use statistics that approximately describe cross-linked polymerized complex molecules. Unlike plastics, cellulose, lignin, petroleum resid, and coal generally have a set of free terminal substituents with a continuous distribution of carbon number attached to the ring structures of the polymer cores. Embodiments can apply a set of special free terminal substituents called side chains (SC) 554a-c, beyond the definition of linear polymer statistics to model the structures. FIG. 5 illustrates an embodiment where each core 551a-c attaches to an additional SC structure 554a-c, respectively. Using the abstraction methodology described in relation to FIG. 5, embodiments (e.g., method 330 at step 331) can employ modified statistics to represent sustainable feedstocks (e.g., the feedstocks 110-118 described hereinabove in relation to FIGS. 1A-E).

Table 1 summarizes the number of cores, ILs, FTILs, and SCs for representing sustainable feedstocks, namely, petroleum resid, coal, lignin, cellulose, and plastics, according to embodiments. Embodiments use a finite number of structural attributes O (30˜500) to describe a nearly infinite number of polymerized complex structures in feedstocks and provide a practical computational method to model chemical processes of those feedstocks. The mathematical details of embodiments include modified statistics that are coded into embodiment's MB attribute modeling (AM) sampling protocol to define molecules represented by the attributes. For instance, in an embodiment, for each feedstock (e.g., resid, coal, lignin, cellulose, and plastics), users can specify a predefined attribute list of core, IL, FTIL, and SC as shown in Table 1. Cluster size of each feedstock may also be specified by users. MB attribute modeling will generate codes of equations shown below (Eq.1 to Eq. 27) in terms of EO format: residuals, jacobians, and sparsities. Such functionality is described below

TABLE 1
Statistics of attributes in sustainable feedstocks

| Feedstock Type | Core | IL | FTIL | SC | Cluster Size |
|---|---|---|---|---|---|
| Petroleum Resid | O(100-200) | O(10) | Few | O(50-100) | 1~3 |
| Coal | O(100) | O(20) | O(20) | O(10) | O(30) |
| Lignin | O(30) | O(30) | O(30) | 0 | O(100) |
| Cellulose | O(10) | O(10) | O(10) | 0 | >>1000 |
| Plastics | O(10) | O(10) | O(10) | 0 | >>1000 |

At first, a parameter called Flory p is introduced into the MB AM sampling model. Flory p is used to describe the polymerized fraction in a mixture of components shown as FIG. 5. Then, the fraction of monomer or island-structure components is represented by Eq. 1.

$$K ( 1 ) = 1 - p \qquad Eq . 1$$

An embodiment assumes the probability of any two cores being bonded together in a complex structure is the same. The fraction of heavier molecules, e.g., dimer and larger, of a given cluster size (cluster size is based on the number of cores in an embodiment represented in FIG. 5) is represented by Eq. 2.

$$K ( n ) = {p}^{n - 1} \cdot ( 1 - p ) \qquad Eq . 2$$

In the case where n is equal to 1, Eq. 2 becomes Eq. 1.

Any individual molecule of a given cluster size is defined by K(n) and multinomial distributions of cores, ILs, SCs and FTILs. For the specific instances of select feedstocks such as resid or coal, embodiments apply a special conditional probability to the first core of dimer and above structures. A heavy core 551a is selected as the first core of any archipelago structures 550 defined in FIG. 5. The heavy core is filtered by structural conditions such as minimum ring number. For example, embodiments can select heavy cores as structures having at least four fused rings for petroleum resid. Other cores in archipelago structures 550 shown in FIG. 5 can be selected from all possible core structures 551a-c. For feedstocks that do not need conditional probability (e.g., plastics, cellulose etc.), embodiments can remove the heavy core conditions and the first core can also be picked from all possible core structures and treated the same as second or later cores in an archipelago structure. Therefore, an archipelago molecule 550 having a cluster size n has 1 heavy core, n−1 cores, n−1 ILs, n SCs and 2 FTILs. There is a predefined list of different structural attributes of cores, SCs, ILs and FTILs for each feedstock from which the feedstock can be selected to define an archipelago molecule in FIG. 5. According to an example embodiment, an archipelago molecule having a cluster size n will assemble one heavy core from predefined core attributes. Then, n−1 core additional core structures are picked from the predefined core attributes. As shown in FIG. 5, the archipelago molecule 550 contains one IL 552 piece between a contingent heavy core 551a and core 551b. At two terminal ends of the archipelago molecule 550, two FTIL structures 553a and 553b are determined from predefined FTIL attributes. Each core and heavy core are attached to one SC 554a-c from predefined SC attributes. The model reads the predefined attributes of cores, ILs, FTILs, and SCs as input information and, then, populates a set of archipelago molecules. Compositions of those molecules are estimated by codes of Eq. 3 to Eq. 7. The codes of those equations are automatically written in equation oriented format including residuals, jacobians, and sparsities. To simplify the problem, an embodiment assumes there are uniform probability distributions of those predefined structural attributes (Cores, SCs, ILs, and FTILs) respectively to be assembled in each structural moiety (1^{st} of cores, 2^{nd} of cores, . . . , n−1th of cores, 1^{st} of SCs, 2^{nd} of SCs, . . . , nth of SCs, 1^{st} of ILs, 2^{nd} of ILs, . . . , n−1th of ILs, 1^{st} of FTILs and 2^{nd} of FTILs) in those archipelago molecules. As a result, the mole fraction of an archipelago molecule is shown in Eq. 3:

$${f}_{a r c} = K ( n ) \cdot {f}_{h v y c o r e} \cdot \frac{( n - 1 ) !}{\prod m !} \cdot \prod {f}_{c o r e}^{m} \cdot \frac{n !}{\prod l !} \cdot \prod {f}_{S C}^{l} \cdot \frac{( n - 1 ) !}{\prod k !} \cdot \prod {f}_{IL}^{k} \cdot \frac{2 !}{\prod j !} \cdot \prod {f}_{FTIL}^{j} \qquad Eq . 3$$

f_{hvycore} is the fraction of heavy core in that molecule and Σf_{hvycore}=1.

$$\frac{( n - 1 ) !}{\prod m !} \cdot \prod {f}_{c o r e}^{m}$$

is the distributed fraction of the other n−1 cores for a given core configuration. Σf_{core}=1, f_{core} is the distribution of cores in a feedstock and m is the number of a given core in that archipelago molecule.

$$\frac{n !}{\prod l !} \cdot \prod {f}_{S C}^{l}$$

is the distributed traction of n SCs for a given SC configuration. Σf_{SC}=1, f_{SC} is the distribution of SCs in a feedstock and 1 is the number of a given SC in that archipelago molecule.

$$\frac{( n - 1 ) !}{\prod k !} \cdot \prod {f}_{IL}^{k}$$

is the distributed fraction of n−1 ILs for a given IL configuration. Σf_{IL}=1, f_{IL} is the distribution of ILs in a feedstock and k is the number of a given IL in that archipelago molecule.

$$\frac{2 !}{\prod j !} \cdot \prod {f}_{FTIL}^{j}$$

is the distributed fraction of 2 FTILs for a given FTIL configuration. Σf_{FTIL}=1, f_{FTIL} is the distribution of FTILs in a feedstock and j is the number of a given FTIL in that archipelago molecule.

To describe a hybrid system that has both individual molecules (IM) and heavy molecules defined in FIG. 5 by the above statistics, an embodiment introduces an additional parameter f_{AM} as the fraction of molecules defined by MB AM statistics. In such an embodiment, the total portion of all archipelago molecules (dimer and above) is shown as Eq 4:

$${f}_{arc\_total} = p \cdot {f}_{AM} \qquad Eq . 4$$

The fraction of an archipelago molecule in such a hybrid system is shown as Eq. 5:

$${f}_{arc}^{′} = {f}_{arc} \cdot p \cdot {f}_{AM} \qquad Eq . 5$$

The portion of total island molecules (monomer) is shown in Eq. 6:

$${f}_{arc\_total} = ( 1 - {f}_{A M} ) + ( 1 - p ) \cdot {f}_{A M} \qquad Eq . 6$$

The first term in Eq. 6 is the portions of all individual molecules that are not defined by an MB AM sampling model. Embodiments may describe molecules individually in the hybrid system as monomers. In general, embodiments have around O(50˜500) such individual molecules in process models. The second term in Eq. 6 is the portion of all island molecules defined by MB AM sampling model (cluster size is 1) and the contribution of an isomer to/from that island structured model is shown in Eq.7:

$${f}_{island} = ( 1 - p ) \cdot {f}_{AM} \cdot {f}_{core} \cdot {f}_{SC} \cdot \frac{2 !}{\prod j !} \cdot \prod {f}_{FTIL}^{j} \cdot {f}_{isom} \qquad Eq . 7$$

fᵢₛₒₘ is the fraction of an isomeric molecule having a given Core, SC, and the configuration of FTILs.

Embodiments can use a limited set of IMs, cores, ILs, SCs, and FTILs to set up a hybrid attribute-based model and describe the nearly infinite components in sustainable feedstocks and small molecules derived from the feedstocks in any chemical processes using Eq. 1 to Eq. 7.

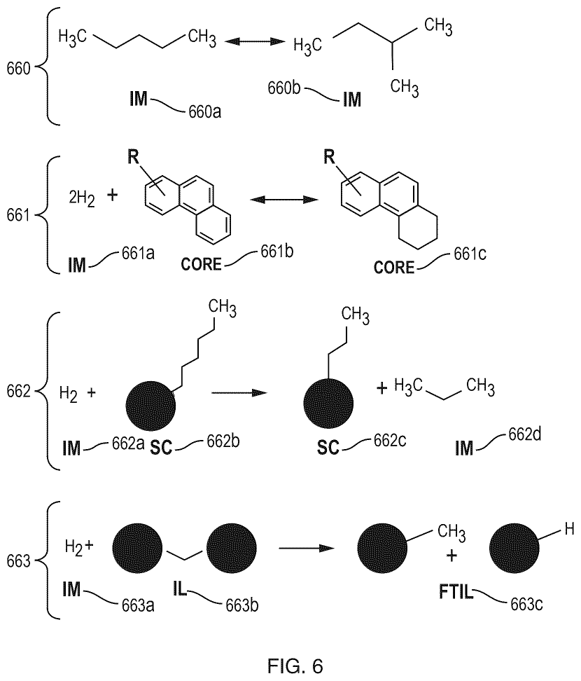

To model the reactions of sustainable feedstocks via the attributes described in the embodiments and IMs, embodiments can utilize a new enhanced attribute reaction model in Aspen MB EORXR. FIG. 6 shows an example of the reactions in a hydrocracker process that can be simulated using embodiments.

FIG. 6 depicts an example of attribute reactions in a hydrocracking model. As shown in FIG. 6, there are four types of reactions 660-663 that are modeled in the reactor. The first reaction 660, is the reaction of individual molecules 660a and 660b, the isomerization of n-pentane. The second reaction 661 is the reaction of cores: phenanthrene ring 661b is saturated to a tetrahydro phenanthrene ring 661c using the individual molecule of hydrogen 661a. The third reaction 662 is the reaction of SCs: a SC of six carbons 662b is cracked to another shorter SC of three carbons 662c and a propane 662d using the individual molecule hydrogen 662a. The reactions of cores and SCs are processed independently. SCs and cores are not allowed to be written in the same reaction. In addition, the stoichiometric coefficient of core or SC in the left-hand side (LHS) must be equal to that of core or SC in the right-hand side (RHS). The fourth reaction 663 is the reaction of IL and FTIL: a carbon bridge 663b is broken down to two substituents: methyl and hydrogen 663c using the individual molecule hydrogen 663a. Only the reactions of ILs and TILs are important to highly polymerized aggregated molecules. IMs can appear in all four reaction types: 660, 661, 662, and 663. As result, the mass balance equations in a reactor can be written as Eq. 8 to Eq. 16.

$$\frac{d {F}_{{IM}_{i}}}{d V} = {\sum }_{j} {a}_{j} \cdot {r}_{{IM}_{j}} + {\sum }_{k} {b}_{k} \cdot {r}_{C o r {e}_{k}} + {\sum }_{l} {c}_{l} \cdot {r}_{S {C}_{l}} + {\sum }_{l} {d}_{m} \cdot {r}_{{IL}_{m}} + {\sum }_{l} {e}_{n} \cdot {r}_{{FTIL}_{n}} \qquad Eq . 8$$

$$\frac{d {F}_{{Core}_{k}}}{d V} = {\sum }_{k} {bb}_{k} \cdot {r}_{{Core}_{k}} \qquad Eq . 9$$

$$\frac{d {F}_{S {C}_{l}}}{d V} = {\sum }_{l} c {c}_{l} \cdot {r}_{{SC}_{l}} \qquad Eq . 10$$

$$\frac{d {F}_{{IL}_{m}}}{d V} = {\sum }_{l} d {d}_{m} \cdot {r}_{{IL}_{m}} \qquad Eq . 11$$

$$\frac{d {F}_{{FTIL}_{n}}}{d V} = {\sum }_{l} e {e}_{n} \cdot {r}_{{FTIL}_{n}} \qquad Eq . 12$$

$$\frac{p}{1 - p} = \frac{2 \cdot \sum {F}_{IL}}{\sum {F}_{FTIL}} \qquad Eq . 13$$

$${F}_{{total}_{IM}} = {\sum }_{i} {F}_{{IM}_{i}} = 1 - {f}_{A M} + ( 1 - p ) \cdot {f}_{A M} \qquad Eq . 14$$

$${F}_{total} = {F}_{{total}_{IM}} + {F}_{{total}_{Att}} \qquad Eq . 15$$

$${F}_{{total}_{Att}} = p \cdot {f}_{A M} \qquad Eq . 16$$

aⱼ, bₖ, cₗ, dₘ, eₙ are the stoichiometric coefficients of IM in reactions
bbₖ are the stoichiometric coefficients of Core in reactions
ccₗ are the stoichiometric coefficients of SC in reactions
ddₘ are the stoichiometric coefficients of IL in reactions
eeₙ are the stoichiometric coefficients of FTIL in reactions

Eq. 8 to Eq. 12 are ordinary differential equations (ODEs) for IMs, Cores, SCs, ILs and FTILs, respectively, and embodiments can use the ODEs to update the individual molecules and attributes in Cores, SCs, ILs and FTILs during the reactor integration. Reactor integration, according to an embodiment, refers to a classic mathematical definition of integration, thus integration along the reactor to solve an equation. According to an embodiment, for each iteration point in the calculation of reactor integration, Eq. 13 is used to update the Flory p of MB AM sampling statistics and, then, an embodiment can use Eq. 14 to Eq. 16 to evaluate the total mole flow rate of the mixture in the reactor.

The energy balance equation and the momentum balance equation of that new hybrid ARM model are similar to the conventional equations in MB EORXR and are shown in Eq.17 and Eq.18 respectively.

$$\frac{dT}{dV} = \frac{1}{{F}_{total} \cdot Mix Cp} {V}_{p} \sum {r}_{i} \cdot ( - Δ {H}_{{rxn}_{i}} ) + UA ( T - {T}_{c} ) \qquad Eq . 17$$

T is the temperature in the reactor bed
Fₜₒₜₐₗ is the total mole flow rate in the reactor bed
Vₚ is the volume of the reactor bed
rᵢ is the reaction rate of reaction i and ΔHᵣₓₙ_{ᵢ} is the enthalpy change of reaction i
UA is the heat transfer coefficient to the environment and T_{c} is the environment temperature

For an adiabatic reactor (e.g., a hydrocracker (HCR)), the second term of Eq. 17 can be ignored

$$\begin{matrix}\frac{dP}{dZ} = - f \cdot \frac{ρ \cdot {u}_{s}^{2}}{{d}_{p}} \\ f = \frac{1 - ε}{{ε}^{3}} \cdot ( 1.75 a + 150 b \frac{( 1 - ε )}{Re} )\end{matrix} \qquad Eq . 18$$

P is the pressure in the reactor bed
ρ is the density of the stream
μₛ is the superficial velocity
dₚ is the diameter of the catalyst particle in the reactor bed
ε is the void fraction of a reactor bed

f is the friction factor

Re is Reynold number

a and b are turbulent and laminar correction coefficients. By default, a=b=1

The effluent of the reactor may have a list of product IMs, Cores, ILs and FTILs and then an embodiment can use Eq.1 to Eq.7 to resample the list of product IMs, Cores, ILs, and FTILs back to individual molecular components in the product stream if necessary. According to an embodiment, the MB AM reactor model of polymerized complex mixtures is incorporated into MB EORXR builder. As a result, the residuals, analytical jacobians and sparsity patterns of the model Eq.1 to Eq.18 are automatically generated.

In addition to reactor models, embodiments can also consider a property estimation model in terms of the MB AM sampling model, which estimates properties of each sampled archipelago molecule and bulk properties of the mixture in terms of attributes and individual molecules. Instead of calculating properties from individual molecules, embodiments can calculate properties of archipelago molecules defined in FIG. 5 from properties of the attributes (Cores, SCs, ILs and FTILs). Based on these essential molecular structures, the properties of the feedstock are then extrapolated from the computational model. For properties applicable to linear mixing, an embodiment can directly calculate those attribute-defined molecules using linear mixing rules, in accordance with principles known to those of skill in the art. For properties that are not applicable to linear mixing, an embodiment can employ a group contribution method and apply the linear mixing rule to estimate the mixed functional group of a given property from the functional groups of the attributes and further estimate the property. Table 2 shows a list of properties used in attribute based models (e.g., a MB AM model) according to an embodiment.

TABLE 2
The molecular properties used in MB AM model

| | Directly Linear Mixing |
|---|---|
| Property Name | Rule |
| MW | Yes |
| CarbonNum | Yes |
| HydrogenNum | Yes |
| SideChainNum | Yes |
| AromRingNum | Yes |
| NaphRingNum | Yes |
| ThphRingNum | Yes |
| PyrrolicRingNum | Yes |
| PyrridenicRingNum | Yes |
| TotalSulfur | Yes |
| TotalNitrogen | Yes |
| TotalOxygen | Yes |
| AromCarbonNum | Yes |
| NaphCarbonNum | Yes |
| ParCarbonNum | Yes |
| Naph6Ring | Yes |
| Naph5Ring | Yes |
| Tb | No |
| Tm | No |
| Density | No |
| Gform | Yes |
| Cp_a | Yes |
| Cp_b | Yes |
| Cp_c | Yes |
| Cp_d | Yes |
| Hvap | Yes |
| HfLiq | Yes |
| CpLiq_a | Yes |
| CpLiq_b | Yes |
| CpLiq_c | Yes |

Embodiments can sample all possible archipelago molecules as defined in FIG. 5 individually and there will be an infinite number of molecules if such embodiments consider the cluster size from 2 to infinity (cluster size 1 is considered as an island molecule or monomer). However, in practice, embodiments do not need to calculate all possible molecules, rather, embodiments can sample out a finite number of molecules ranging from cluster size 2 to nc and keep the remaining part as an “unsampled” mixed component. Here nc is a critical cluster size number that is the maximum cluster size of individual archipelago molecules required for a given chemical process or property estimation (e.g., distillation curve, solubility curve, etc.). Embodiments can apply Equations 1 to 3 and the linear mixing rule (i.e., a rule where a bulk property is calculated by summing the product of the composition of the individual molecules by the property of the molecules) to estimate the properties of sampled molecules and unsampled components by a selected a critical cluster size number (nc) for the portion of all archipelago molecules.

To implement such functionality, first, an embodiment counts average properties of archipelago molecules, e.g., all archipelago molecules. The summation of mole fractions of archipelago molecules is equal to p as shown in Eq. 19.

$${\sum }_{n = 2}^{∞} K ( n ) = {p}^{n - 1} \cdot ( 1 - p ) = p \qquad Eq . 19$$

The average properties or average property functional groups of all predefined cores, SCs, ILs, FTILs, and heavy cores are shown in Eq.20.

$$\begin{matrix}\overline{{Prop}_{ι}^{core}} = \sum {f}_{core} \cdot {Prop}_{i}^{core} \\ \overline{{Prop}_{ι}^{SC}} = \sum {f}_{SC} \cdot {Prop}_{i}^{SC} \\ \overline{{Prop}_{ι}^{IL}} = \sum {f}_{IL} \cdot {Prop}_{i}^{IL} \\ \overline{{Prop}_{ι}^{FTIL}} = \sum {f}_{FTIL} \cdot {Prop}_{i}^{FTIL} \\ \overline{{Prop}_{ι}^{hvycore}} = \sum {f}_{core} \cdot {Prop}_{i}^{hvycore}\end{matrix} \qquad Eq . 20$$

P̅r̅o̅p̅ᵢ̅^̅{̅c̅o̅r̅e̅}̅, P̅r̅o̅p̅ᵢ̅^̅{̅S̅C̅}̅, P̅r̅o̅p̅ᵢ̅^̅{̅I̅L̅}̅, P̅r̅o̅p̅ᵢ̅^̅{̅F̅T̅I̅L̅}̅, P̅r̅o̅p̅ᵢ̅^̅{̅h̅v̅y̅c̅o̅r̅e̅}̅ are the average property or the average property functional group of all predefined cores, SCs, ILs, FTILs, and heavy cores for a given property I, respectively

To continue, for a mixture of archipelago molecules with a cluster size ranging from 2 to infinity, average property or average property functional group for a given property i is estimated as Eq. 21

$$\overline{{Prop}_{ι}} = \frac{( \begin{matrix}{\sum }_{n = 2}^{∞} K ( n ) \cdot \overline{{Prop}_{ι}^{hvycore}} + {\sum }_{n = 2}^{∞} ( n - 1 ) \cdot K ( n ) \cdot \overline{{Prop}_{ι}^{core}} + \\ {\sum }_{n = 2}^{∞} ( n - 1 ) \cdot K ( n ) \cdot \overline{{Prop}_{ι}^{IL}} + {\sum }_{n = 2}^{∞} ( n \cdot K ( n ) \cdot \overline{{Prop}_{ι}^{SC}} ) + {\sum }_{n = 2}^{∞} 2 \cdot K ( n ) \cdot \overline{{Prop}_{ι}^{FTIL}}\end{matrix} )}{{\sum }_{n = 2}^{∞} K ( n )} \qquad Eq . 21$$

The property value or the property functional group of a given property i for a sampled archipelago molecule via Eq. 1 to Eq. 3 can be calculated by explicitly counting the property value of each attribute (core, SC, IL, FTIL and heavy core) contained in that molecule as shown by Eq. 22.

$${Prop}_{i} = {Prop}_{i}^{hvycore} + \sum m \cdot {Prop}_{i}^{core} + \sum l \cdot {Prop}_{i}^{SC} + \sum k \cdot {Prop}_{i}^{IL} + \sum j \cdot {Prop}_{i}^{FTIL} \qquad Eq . 22$$

Propᵢ is the property value or the property functional group of a given property i for a sampled archipelago molecule
m is the number of a given core in that archipelago molecule
l is the number of a given SC in that archipelago molecule
k is the number of a given IL in that archipelago molecule
j is the number of a given FTIL in that archipelago molecule

The properties of a set of sampled archipelago molecules having a cluster size ranging from 2 to nc can be calculated by Eq. 22 and embodiments can estimate the property of the “unsampled” mixture by excluding each individual sampled molecule from the entire mixture of archipelago molecules. Derived from Eq. 2, Eq. 3, and Eq. 22, embodiments can use Eq. 23 to calculate the average property or the average property functional group of a given property i for “unsampled” components.

$${Prop}_{i}^{unsampled} = ( p \cdot \overline{{Prop}_{ι}} - \sum {f}_{arc} \cdot {Prop}_{i} ) / ( p - \sum {f}_{arc} ) \qquad Eq . 23$$

For the properties that are not applicable for linear mixing directly, embodiments can use the functional group of an individual sampled archipelago molecule, the unsampled component, and the average mixture to calculate corresponding properties by a given group contribution method. Using Eq. 19 to Eq. 23 and Eq. 2, embodiments can obtain the mole fractions and the properties of a set of sampled archipelago molecules and the remaining part, the unsampled component. If embodiments have a moderate number of predefined attributes (cores, SCs, ILs and FTILs), embodiments can use sampled molecules and unsampled components directly as thermodynamic components for determining properties of a chemical feedstock undergoing separation processes (vapor-liquid equilibrium (VLE), liquid-liquid equilibrium (LLE), solid-liquid equilibrium (SLE), etc.) and the properties of those thermodynamic components can be evaluated using methods described herein. However, there is a computational combinatorial problem if the embodied system (i.e., feedstock) has a large number (e.g., 10) of predefined attributes (cores, SCs, ILs, FTILs). Amongst other examples, this is often the case for resid and coal. If a represented system has a large number of predefined attributes, an embodiment can extend an MB Rigorous Reversible Lumping (RRL) method (WO 2023/193172) to those archipelago molecules to obtain a limited number of thermodynamic components for simulating separation processes. In such an embodiment, initially, nc is controlled to a practical number, e.g., O (20) to O (50). Then, such an embodiment can apply a cluster analysis to each attribute type (core, SC, IL, FTIL) individually and obtain a set of attribute groups per each attribute type (core, SC, IL, FTIL). Instead of sampling individual molecules from individual attributes (cores, SCs, ILs, FTILs) directly, such an embodiment can use equations similar to Eq. 2 and Eq. 19 to Eq. 23 to obtain a set of sampled archipelago lumps and unsampled components as shown by Eq. 24.

$${f}_{arc - group} = K ( n ) \cdot {f}_{hvycore - group} \cdot \frac{( n - 1 ) !}{\prod mm !} \cdot \prod {f}_{core - group}^{mm} \cdot \frac{n !}{\prod ll !} \cdot \prod {f}_{SC - group}^{ll} \cdot \frac{( n - 1 ) !}{\prod kk !} \cdot \prod {f}_{IL - group}^{kk} \cdot \frac{2 !}{\prod jj !} \cdot \prod {f}_{FTIL - group}^{jj} \qquad Eq . 24$$

f_{hvycore-group} is the fraction of heavy core group in that molecule and Σf_{hvycore-group}=1

$$\frac{( n - 1 ) !}{\prod mm !} \cdot \prod {f}_{core - group}^{mm}$$

is the distributed group fraction of other n−1 cores for a given core group configuration

Σf_{core-group}=1, f_{core-group} is the distribution of core groups in a feedstock and mm is the number of a given core group in that archipelago molecule

$$\frac{n !}{\prod ll !} \cdot \prod {f}_{SC - group}^{ll}$$

is the distributed group fraction of n SCs for a given SC group configuration

Σf_{SC-group}=1, f_{SC-group} is the distribution of SC groups in a feedstock and ll is the number of a given SC group in that archipelago molecule

$$\frac{( n - 1 ) !}{\prod kk !} \cdot \prod {f}_{IL - group}^{kk}$$

is the distributed group fraction of n−1 ILs for a given IL group configuration

Σf_{IL-group}=1, f_{IL} is the distribution of ILs in a feedstock and kk is the number of a given IL group in that archipelago molecule

$$\frac{2 !}{\prod jj !} \cdot \prod {f}_{FTIL - group}^{jj}$$

is the distributed group fraction of 2 FTILs for a given FTIL group configuration

Σf_{FTIL-group}=1, f_{FTIL-group} is the distribution of FTILs in a feedstock and jj is the number of a given FTIL group in that archipelago molecule

The average properties or average property functional groups of all attribute groups of cores, SCs, ILs, FTILs, and heavy cores are shown in Eq. 25.

$$\begin{matrix}{Prop}_{i}^{core - group} = \sum {f}_{core - group} \cdot {Prop}_{i}^{core} \\ {Prop}_{i}^{SC - group} = \sum {f}_{SC} \cdot {Prop}_{i}^{SC} \\ {Prop}_{i}^{IL - group} = \sum {f}_{IL} \cdot {Prop}_{i}^{IL} \\ {Prop}_{i}^{FTIL - group} = \sum {f}_{FTIL} \cdot {Prop}_{i}^{FTIL} \\ {Prop}_{i}^{hvycore - group} = \sum {f}_{core} \cdot {Prop}_{i}^{hvycore} \\ {Prop}_{i}^{core - group} , {Prop}_{i}^{SC - group} , {Prop}_{i}^{IL - group} , {Prop}_{i}^{FTIL - group} ,\end{matrix} \qquad Eq . 25$$

Propᵢ^{hvycore-group} are the property or the property functional group of a given attribute group (cores, SCs, ILs, FTILs, and heavy cores) for a given property i respectively.

The property value or the property functional group of a given property i for a sampled archipelago lump defined by Eq. 24 can be calculated by explicitly counting the property value of each attribute group (core, SC, IL, FTIL and heavy core) configured in that molecule, as shown in Eq. 26.

$${Prop}_{i}^{group} = {Prop}_{i}^{hvycore - group} + \sum mm \cdot {Prop}_{i}^{core - group} + \sum ll \cdot {Prop}_{i}^{SC - group} + \sum kk \cdot {Prop}_{i}^{IL - group} + \sum jj \cdot {Prop}_{i}^{FTIL - group} \qquad Eq . 26$$

Propᵢ^{group} is the property value or the property functional group of a given property i for a sampled archipelago molecule
mm is the number of a given core group in that archipelago lump
ll is the number of a given SC group in that archipelago lump
kk is the number of a given IL group in that archipelago lump
jj is the number of a given FTIL group in that archipelago lump

The properties of a set of sampled archipelago lumps having the cluster size ranging from 2 to nc can be calculated by Eq. 26 and embodiments can estimate the property of the “unsampled” mixture by excluding each individual sampled lump from the entire mixture of archipelago molecules. Derived from Eq. 21, Eq. 24, and Eq. 26, embodiments can use Eq. 27 to calculate the average property or the average property functional group of a given property i for the “unsampled” component.

$${Prop}_{i}^{unsampled} = ( p \cdot \overline{{Prop}_{ι}} - \sum {f}_{arc} \cdot {Prop}_{i}^{group} ) / ( p - \sum {f}_{arc - group} ) \qquad Eq . 27$$

The number of attribute groups (core, SC, IL and FTIL) can be limited to a practical number O (10) based on user input, e.g., requirements. Nonetheless, embodiments can still use O (100) attribute thermodynamic lumps to describe an archipelago system that has a large number of predefined attributes (Core, SC, IL, and FTIL). Then, an internal distribution of individual attributes in a given attribute group remains intact before and after a thermodynamic separation modeled by those attribute lumps. As a result, embodiments still use the MB RRL method to maintain the full molecular information of that archipelago system while using affordable computational resources.

To summarize, an embodiment expands on the Aspen MB EO modeling framework described in WO 2023/193172 with the new attribute representations described hereinabove in relation to Eq. 1 to Eq. 27 to model complex aggregated mixtures (i.e., chemical feedstocks), such as those illustrated in FIGS. 1A-E. The functional blocks in the Aspen MB AM framework that can be utilized in some aspects of embodiments are listed in Table 3.

TABLE 3
MB EO Blocks in Aspen MB ARM Framework

| Block List | Comments |
|---|---|
| MB EOR XR | EO Kinetics Reactor bed |
| MB Conversion | EO Stoichiometric based |
| Reactor | Conversion Reactor |
| MB | Transfer MB Species to/from |
| Lumper/Delumper | LLE/VLE hypos via RRL |
| MB Basic Flash | VLE, LLE, and SLE Flash and Short |
| | cut distillation (SCD) |
| MB Attribute | Mapping individual molecules from |
| Molecule Mapper | archipelago mixtures |
| MB Property | Estimate bulk properties |
| Calculator | |
| MB Mixer | Vapor phase mixer: e.g., quench |
| | mixer/splitter |
| MB Component | H₂S purge, NH₃ remover |
| splitter | |
| MB Feed Adjustor | Feed composition modeler |

The reactor block (“MB Conversion Reactor) listed in Table 3 can be used to model chemical reactions for complex polymerized mixtures. Embodiments can use MB basic flash or transfer to appropriate thermodynamics lumps via MB lumper/delumper and apply to Aspen HYSYS or Aspen Plus to model separation processes among archipelago mixtures (e.g., placing an MB model in an Aspen HYSYS or Aspen Plus flowsheet and solving within that flowsheet). The MB attribute molecule mapper allows embodiments to convert some portions of archipelago mixtures to individual molecules for given chemistries (e.g., polymer pyrolysis). Embodiments can use MB property calculator to estimate bulk properties from archipelago mixtures. In addition, embodiments also can apply MB mixer, MB component splitter and MB feed adjustor to finish process operations in an Aspen EO sub flowsheet (e.g., another flowsheet, but with hidden details at a top level such that the top level sees a single block, but once the sub flowsheet is opened, then multiple unit operations can be observed). In an embodiment, the blocks listed in Table 3 are coded in Aspen EO mode, and the code, including residuals, jacobians, and sparsities is automatically generated by MB builder when users supply their own species and reaction paths. Embodiments can use the blocks in Table 3 and leverage other unit operations (unit ops; e.g., heaters, flow splitters, reactors, distillation columns, flash units, etc.) in Aspen simulators to simulate and optimize a wide range of models of sustainable feedstocks, such as those shown in FIGS. 1A-E.

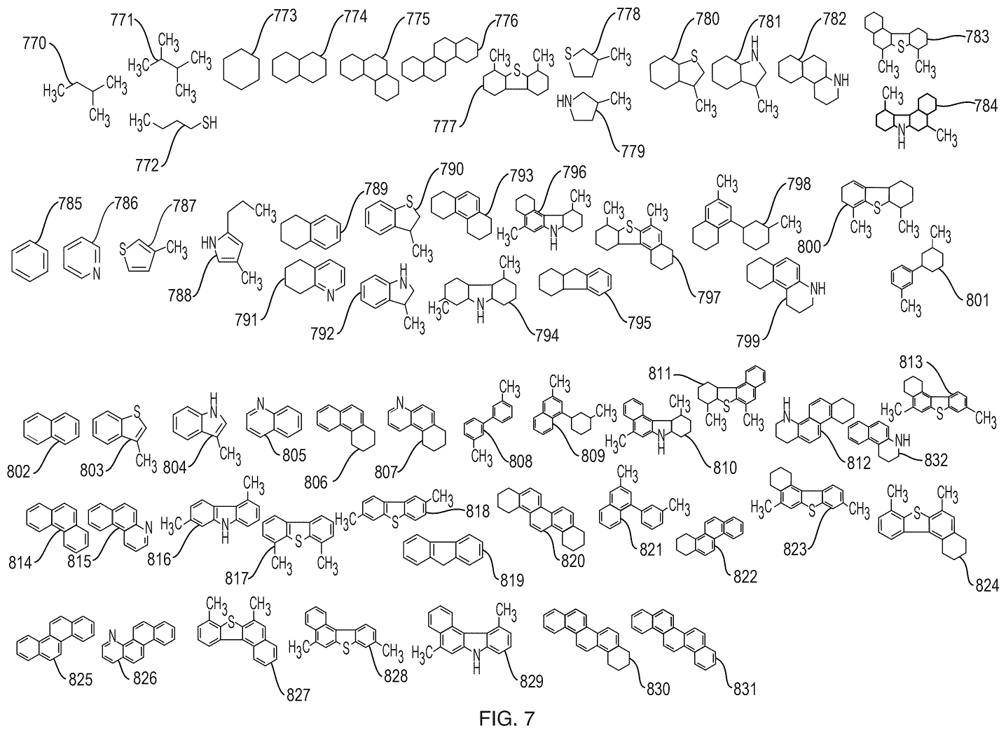

Hereinbelow, example embodiment implementations are described. The first example implementation described below applies an embodiment to petroleum resid. Unlike previous work (e.g., the functionality described U.S. Patent Publication No. 2021/0217497A1), embodiments do not need to explicitly define archipelago structure heavy molecules as individual molecular types (MT) via juxtapositions of cores and ILs. Thus, embodiments use O (50-100) cores and O (10) ILs to describe even heavier resid structures that have a cluster size greater than 3 compared to existing methods that use O (100-1000) MTs to describe resid structures having a cluster size of 1 to 3. FIG. 7 shows a library of cores (770-831) an example embodiment (e.g., implemented in a new Aspen MB AM framework) uses to describe petroleum heavy resid.

In such an embodiment, experimental data from analytical chemistries shows that the number of aromatic rings in aggregated resid molecules is no less than 4. Therefore, such an embodiment applies conditional probability and picks the core structures in FIG. 7 having a ring number greater than or equal to 4 as the first core (heavy core) of archipelago resid molecules and other cores attached in the same resid molecules can be selected from any ring structures in FIG. 7. Reactions of those cores are similar to common hydroprocessing chemistries that individual molecules undergoing different reaction types such as but not limited to saturation, hydrodesulfurization, hydodenitrogenation, and ring opening reactions.

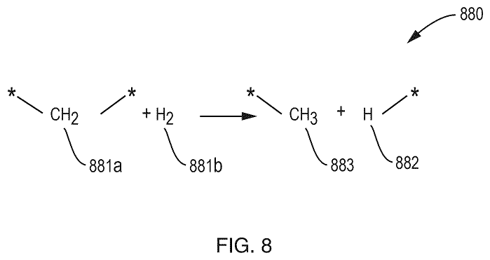

The IL structures in this resid example can be described as a limited number (O (10)) of “bridge” structures such as C bridge (—C—), sulfur bridge (—S—), and a bond bridge (—). An important reaction for ILs is inter-core-linkage cracking that “depolymerizes” the clusters of archipelago complex molecules in resid as illustrated in FIG. 8. FIG. 8 depicts inter-core linkage cracking 880 of heavy resid where the C bridge 881a is cracked by H₂ 881b to two FTILs: —H 882 and —CH₃ 883.

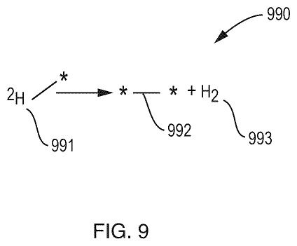

Another reaction type for ILs is a condensation reaction, such as the condensation reaction 990 shown in FIG. 9. FIG. 9 depicts the condensation reaction 990 of heavy resid. The condensation reaction 990 converts two (—H) FTIL 991 to a stable IL, biphenyl bond 992, and hydrogen gas 993. As a result, this reaction 990 shows a mechanism for ring aggregation or coke formation during upgrading of heavy resid.

Due to the nature of structures of petroleum resid, embodiments may impose a special SC attribute type to resid molecules. For example, embodiments can select paraffinic substituents with a carbon number ranging from 1 to 50 as SCs for resid molecules. Upon the classifications of substituent types (paraffinic, branched-paraffinic, olefinic, sulfides, etc.), users can expand details of SCs per each substituent type, so a total number of SCs is O (50˜500). Reactions of those SCs are similar to common cracking chemistries embodiments model for individual molecules: dealkylation and side chain cracking.

Therefore, embodiments can use a total of O (110˜610) attribute structures to describe complex resid molecules and those attributes can be combined with O (50˜1000) individual molecules to describe a resid upgrading process and a downstream resid upgrading process (e.g., naphtha reforming, etc.) in a typical refining flowsheet. In an embodiment, the total species used is O (160˜1610) and using that number of species requires much less computational resources than existing functionality (e.g., U.S. Patent Publication No. 2021/0217497A1) and expands what embodiments can represent to even heavier resid portions (e.g., cluster size greater than 3).

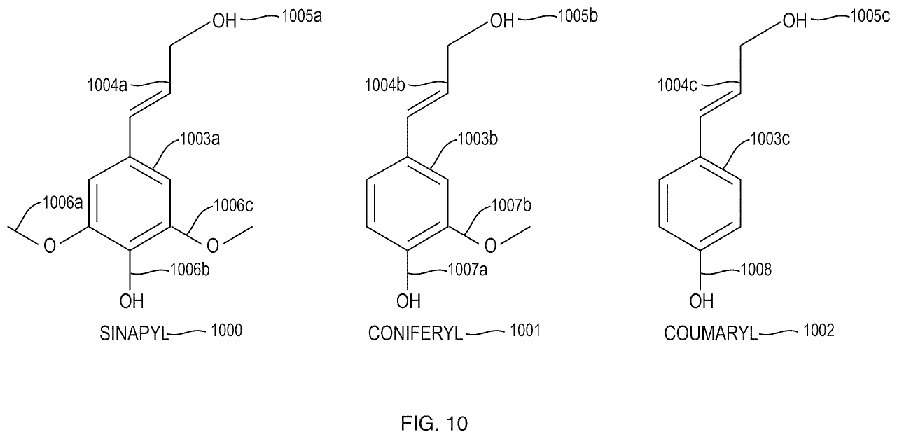

To illustrate another example implementation, a description of applying an embodiment to lignins is provided below. Unlike petroleum resid, lignin is an aggregated macromolecule having highly cross-linked moieties. FIG. 10 depicts typical lignin structures 1000-1002 described by the Freudenberg model. Specifically, FIG. 10 illustrates three well known monomers of lignins, sinapyl 1000, coniferyl 1001, and coumaryl 1002.

Analyzing the monomers 1000-1002 of lignins in FIG. 10, each monomer 1000-1002 can be considered to be a benzene ring 1003a-c with two types of substituents. The substituent on the top side of each benzene ring 1003a-c is a propanoid like structure 1004a-c. Each carbon of those structures 1004a-c can also be linked with an alcohol group (—OH) 1005a-c in the Freudenberg model. The substituents on the bottom side of the benzene rings 1003a-b are a set of 1-3 methoxyl or phenol groups. In sinapyl 1000 the substituents on the bottom side include two methoxy groups 1006a and 1006c and a hydroxyl group 1006b. In coniferyl 1001 the substituents on the bottom side include a methoxy group 1007b and a hydroxyl group 1007a. In coumaryl 1002 the substituents on the bottom side include a hydroxyl group 1008. The esterification of substituents on the top side (e.g., 1004a-c) and the bottom side (e.g., 1006a-c, 1007a-b, and 1008) of benzene rings (e.g., 1003a-c) forms highly cross-linked polymerized lignin structures. To model upgrading of a lignin structure, embodiments consider thermolysis of the lignin complex 1000-1002 shown in FIG. 10. Unlike petroleum resid, a special SC definition is not used to assemble (i.e., represent) the lignin structure, but such an embodiment considers a variety of ILs and FTILs. Except the cracking of the complex of esterified propanoid and methoxyl groups, reactions of methoxyl groups and propanoid groups occur independently. Therefore, an example implementation selects part of a methoxyl group as the cores of the lignin model.

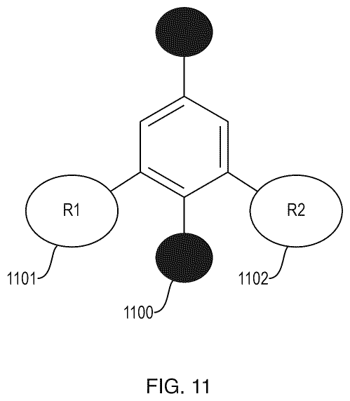

FIG. 11 illustrates a representation 1100 of core structures for lignin in an

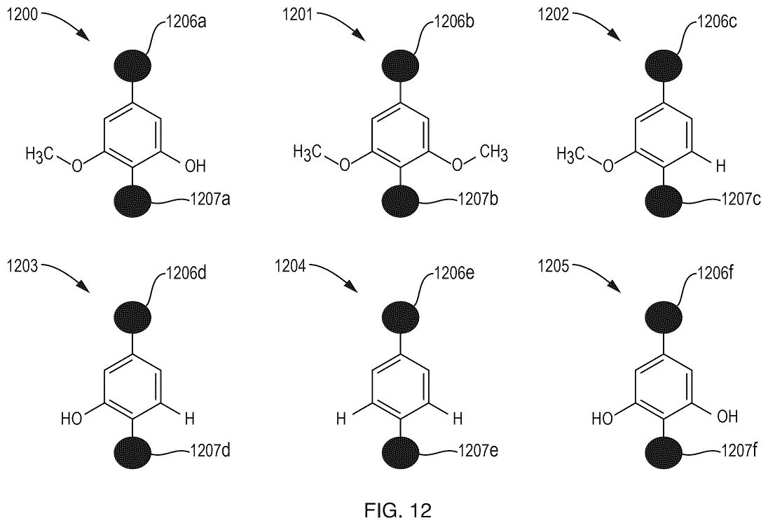

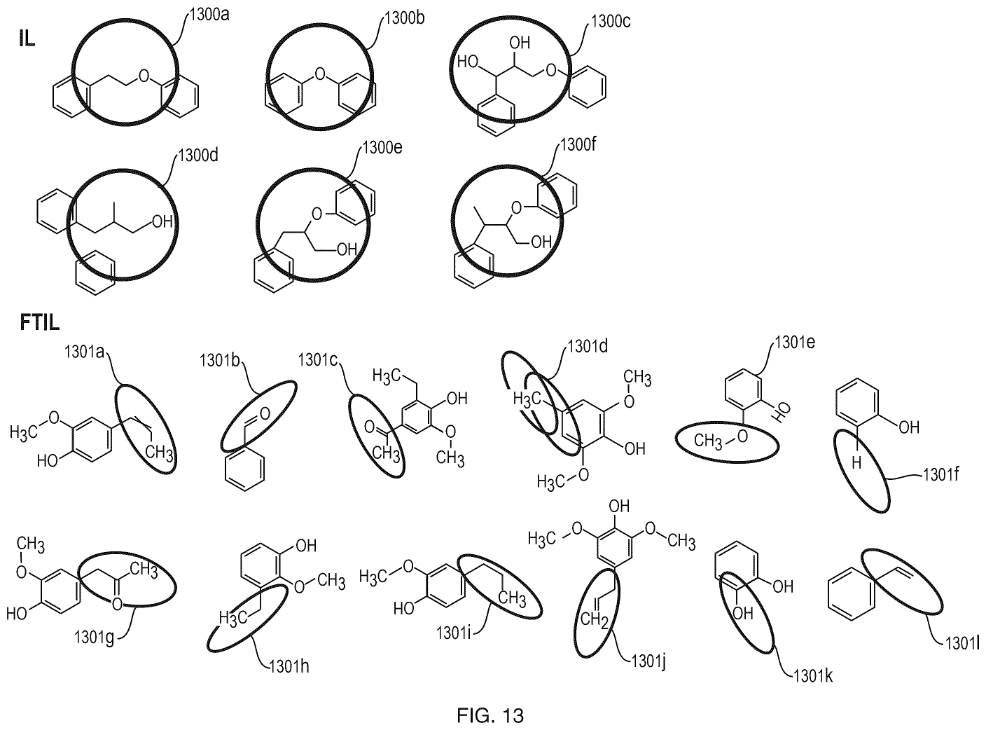

embodiment, wherein R1 1101 and R2 1102 can be altered by any combinations from the substituents: —OH, —OCH₃, and —H. If an embodiment fully enumerates all possibilities, such an embodiment can represent a set of explicit cores of lignin as shown by the representations 1200-1205 in FIG. 12. For the representations 1200-1205 of FIG. 12, top nodes 1206a-f and bottom nodes 1207a-f are connected to either free or esterified propanoid groups and one methoxyl group. According to an embodiment, said free or esterified propanoid groups are defined as ILs 1300a-f as depicted in FIG. 13 and said methoxyl groups are defined as FTILs 1301a-l. Because branched substituents of propanoid and methoxyl groups are explicitly in the cores, ILs, and FTILs of the lignin model, embodiments can use the modified MB AM statistics combining linear polymer statistics and multinomial distributions to describe highly cross-linked lignin structures. Although a model is an approximate representation compared with the full Bethe statistics, such a model is a good approximation for industrial scale engineering simulations.

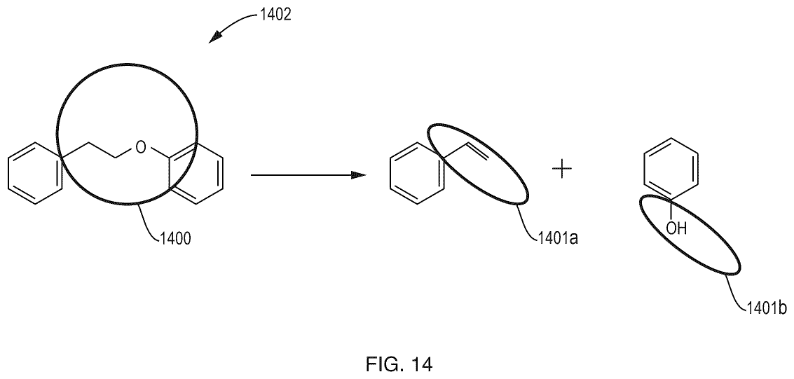

Reaction paths affecting “depolymerization” of lignin structures are the conversions from ILs to FTILs. One such example reaction 1402 is shown in FIG. 14. Specifically, FIG. 14 illustrates a phenethylphenyl ether cracking reaction 1402 that cracks ILs of lignin to FTILs. FIG. 14 shows the IL Phenethylphenyl ether like structure 1400, cracking to one “—OH” phenol-like FTIL 1401b and one styrene-like FTIL 1401a. During lignin thermolysis, an aggregated lignin structure can be pyrolyzed to monomers (island structures) via the ILs' cracking paths.

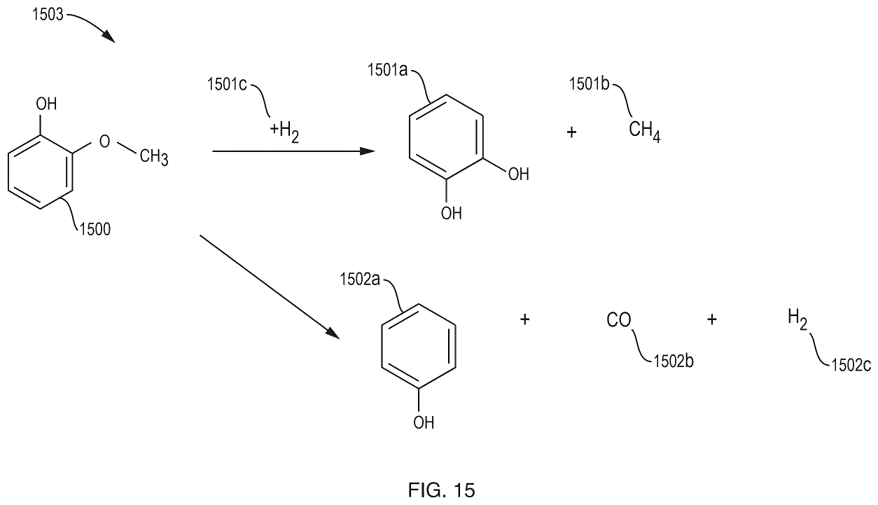

Reactions on a core side can upgrade substituted benzene structures to well-known lignin products and some light gases. FIG. 15 illustrates one such example core side reaction 1503. In the reaction 1503, a guaiacol 1500 is converted in two paths: (i) methane 1501b and catechol 1501a using H₂ 1501c and (ii) phenol 1502a, CO 1502b, and H₂ 1502c shown in FIG. 15.

In an aspect, an embodiment uses the attributes defined in FIG. 12 and FIG. 13, by adjusting the given parameters of p and f_{AM}, so as to use Eq. 1 to Eq. 7 to calculate fractions of the mixture of lignin structures and characterize that mixture by matching the typical properties (e.g., MW) estimated from Eq. 21 to Eq. 27.

To continue, an embodiment can develop a model of lignin thermolysis by creating equations of a reactor following Eq. 8 to Eq. 18. Those equations of a reactor can be automatically utilized in a MB EORXR block via MB builder and users can solve and obtain the solution in Aspen EO. As a result, such an embodiment can determine four kinds of products: light gases (methane, CO, CO₂, H₂), light liquids (H₂O and methanol), main liquid products (a collection of single ring phenolics or benzenes), and a hydrogen-deficient char. The light gases and light liquids are individual molecules in the MB AM framework. The main liquid products are the island molecules (cluster size is 1) upgraded from the reactor and sampled out from the original archipelago mixtures in the MB AM framework. Char is the remainder unsampled archipelago mixture. The products of the lignins can be further processed to get the property estimations or thermodynamic separations via the blocks listed in Table 3.

To illustrate yet another example implementation, a description of applying an embodiment to cellulose is described below. Cellulose is a highly aggregated linear polymerized structure connecting many repeat structure units (monomers) together. Like lignin, special attributes of SCs (such as those described hereinabove in relation to petroleum resid) are also not applicable to cellulose. Major interests to modeling the upgrading process of cellulose is the “depolymerization” of cellulose via thermolysis. Unlike lignin, cores are not interesting attributes of cellulose, the structures of cellulose can be mainly described by IL, the oligomerized repeat units, and FTIL, the unbonded repeat units. Therefore, such an embodiment defines an unchangeable “bond site” as the core to maintain a statistical model.

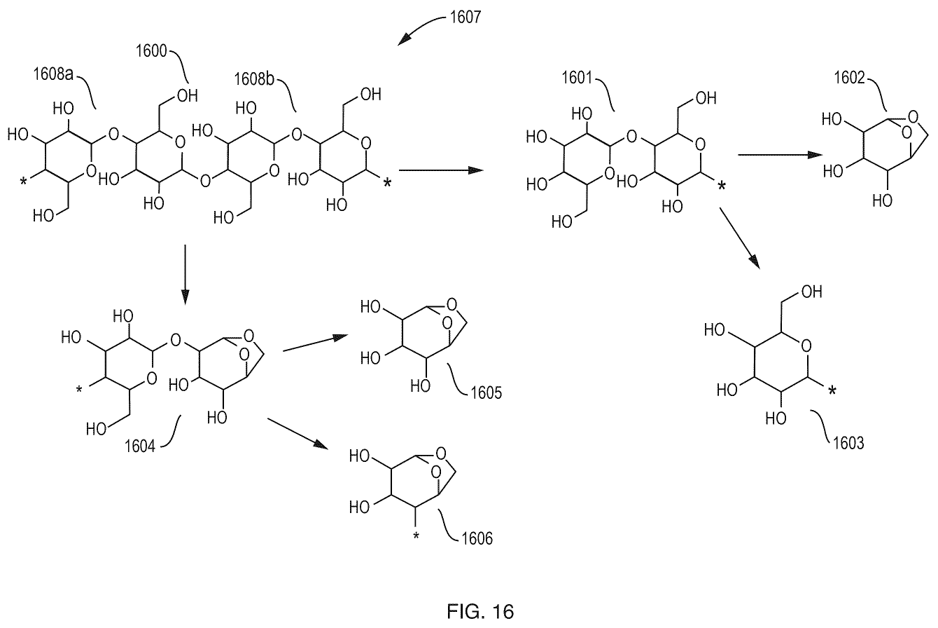

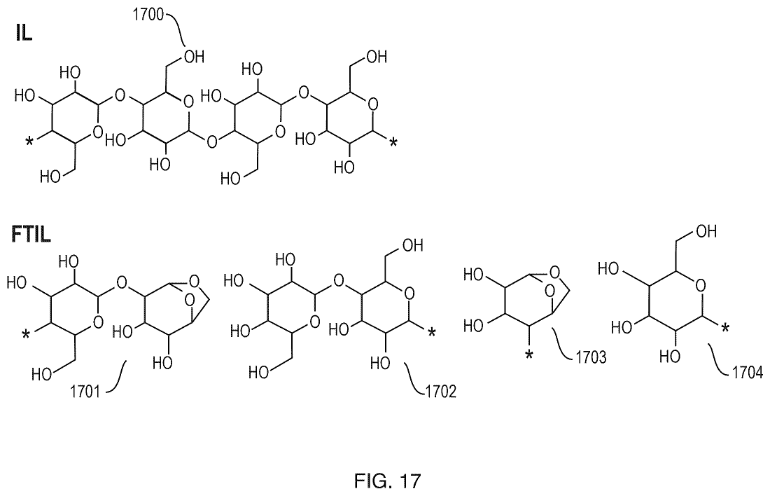

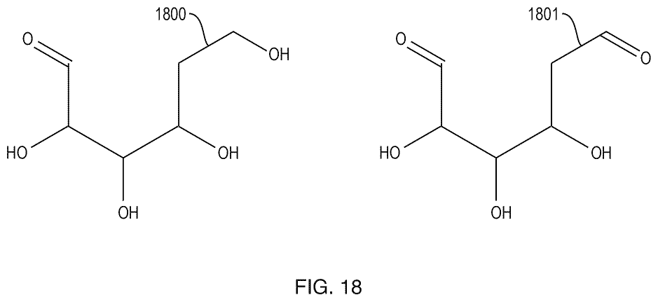

FIG. 16 illustrates a thermolysis reaction 1607 of cellulose 1600. The repeat unit of cellulose 1600 is a pair of beta (1,4) linked D-glucoses 1608a and 1608b. In the thermolysis reaction 1607 of the cellulose 1600, the products are levoglucosan 1602 and glucose 1603 through an intermediate repeat unit 1601. The levoglucosan 1602 is identified as an individual molecule. A 1:1 equal molar ratio of levoglucosan 1605 and levoglucosan 1606 is not cracked from a repeat unit 1604. Instead, two repeat units of the dimer 1600 are the basic structures which can be used to depict the mechanisms of cellulose thermolysis shown in FIG. 16. As a result, an embodiment picks a characteristic length of repeat units as the minimum IL attribute to describe cellulose. In the example described herein, the characteristic length is equal to 2. Given the characteristic length of 2, FIG. 17 illustrates the selected IL structure 1700 for cellulose thermolysis and the selected FTIL structures 1701-1704 (including free terminal glucose and free terminal levoglucosan). In the thermolysis reaction, the structures of glucose and levoglucosan are further cracked to linear small molecules 1800 and 1801 shown in FIG. 18.

Using the attributes (IL 1700, FTILs 1701-1704) illustrated in FIG. 17, by adjusting the given parameters of p and f_{AM}, embodiments can use Eq. 1 to Eq. 7 to calculate fractions of the mixture of original cellulose structures and characterize that mixture by matching the typical properties (e.g., MW) estimated from Eq. 21 to Eq. 27.

To continue, such an embodiment develops a model of cellulose thermolysis by creating the equations of a reactor following Eq. 8 to Eq. 18. In an embodiment, the equations can be automatically generated to a MB EORXR block via MB builder and users can solve and obtain the solution in Aspen EO. An embodiment determines three kinds of products: light gases (CO, CO₂), light liquids (H₂O), and main liquid products (a collection of glucose/levoglucosan derived anhydrosugars). The light gases and light liquids are native individual molecules in a MB AM framework. The main liquid products are the island molecules (cluster size is 1) upgraded from the reactor and sampled out from the original archipelago mixtures in a MB AM framework. The products of cellulose can be further processed for property estimations or thermodynamic separations via the blocks listed in Table 3.

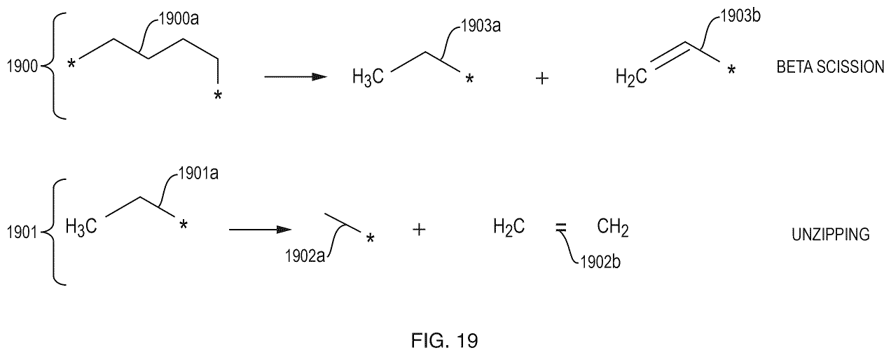

The last example discussed herein is the pyrolysis of plastics. Polyethylene (PE) is used in this example. Similar to cellulose, PE has a typical linear polymer structure. Like lignins and cellulose, no special SC attributes are applied and a “bond site” as a core is used in the model. According to an embodiment, the PE structures are mainly identified in ILs and FTILs in Aspen Technology, Inc.'s (Assignee's) MB AM framework. Unlike the classic moment method to consider the reactions at the mechanistic level, embodiments consider pathway level reactions in the model. FIG. 19 illustrates “unzipping,” or a reverse reaction of polymerization 1901 and regular beta scission 1900 which are two major pathways derived from the radical mechanism of PE pyrolysis. In unzipping 1901, a saturated hydrocarbon derivative, 1901a is broken down into ethylene 1902b and a polyethylene chain that is one monomer shorter 1902a. In beta scission 1900, PE 1900a is broken down into a saturated hydrocarbon derivative 1903a and a an unsaturated hydrocarbon derivative 1903b.

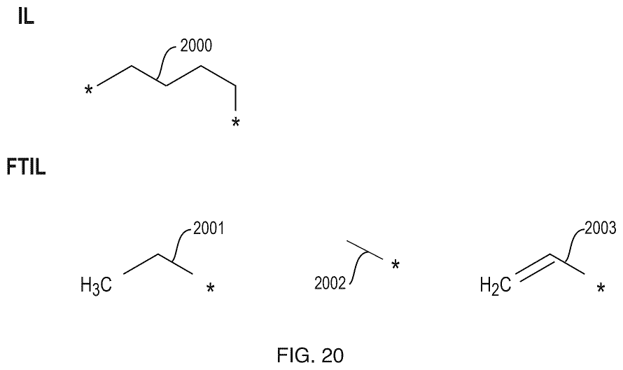

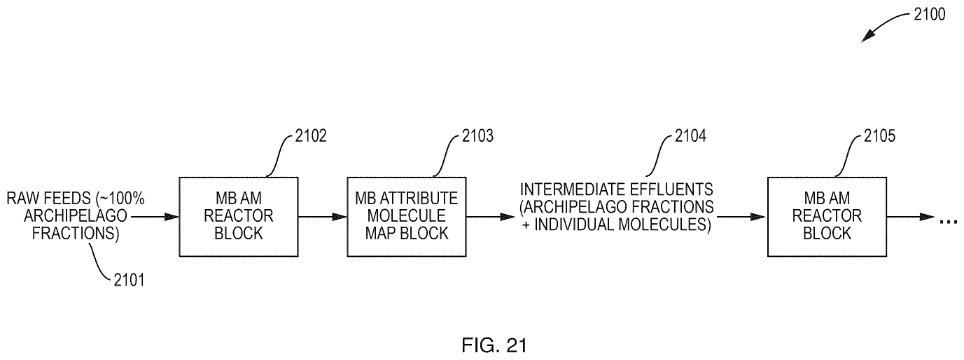

To describe the reaction pathways, a “dimer” of PE 2000 as the basic structure of IL, as shown in FIG. 20, was selected. The FTILs' structures for PE pyrolysis model are derived from the reaction paths 1900 and 1901 in FIG. 19 and the structures of a propane derivative 2001, an ethane derivative 2002, and a propene derivative 2003 are shown in FIG. 20. For low temperature pyrolysis of PE, oligomers of PE (where cluster size is 2 to 40) are high valued liquid products, and the cracking behaviors of those oligomers are important to use. Therefore, an embodiment uses Eq. 1 to Eq. 7 and Eq. 19 to convert oligomers in archipelago mixtures to individual linear molecules that have a carbon number ranging from 2 to 40 (e.g., via a MB attribute molecule mapper) and further explore typical thermal cracking reactions among linear molecules and eventually obtain desired product distributions. The conceptual workflow of such functionality is shown in FIG. 21.

FIG. 21 illustrates the workflow 2100 of PE pyrolysis modeled in MB AM framework. Using the attributes defined in FIG. 20 (IL 2000 and FTILs 2001-2003), by adjusting the given parameters of p and f_{AM}, embodiments use Eq. 1 to Eq. 7 to calculate fractions of the mixture of the original PE structures and characterize that mixture by matching typical properties (e.g., MW) estimated from Eq. 21 to Eq. 27. The characterized PE mixture serves as the feed 2101 in the process 2100.

Then, according to an embodiment, a model of PE thermolysis is developed by creating the equations of a reactor following Eq. 8 to Eq. 18. The reactor equations can be automatically generated to a MB EORXR block 2102 via MB builder. The thermolysis of PE can be decomposed to a set of reactor sections. In such an embodiment, each reactor section is modeled as one MB EORXR block. Between two reactor sections, there is a MB attribute molecule map block 2103 to map oligomers of interest to individual molecules. Reactor sections after the first one will cofeed those archipelago molecules defined by MB AM framework and the individual molecules products from the previous reactor sections to react following the reaction pathways of attributes in FIG. 19 and individual thermal cracking pathways together 2104, resulting in the desired molecular product distribution at the end of reactor outlet 2105. The products of the PE pyrolysis can be further processed for property estimations or thermodynamic separations via the blocks listed in Table 3.

Since the Aspen MB AM framework allows users to describe different archipelago structures (resid, lignin, cellulose, and plastics) and individual molecules together, embodiments can set up a flowsheet including all kinds of examples and, thus, provide users an industrial flowsheet solution to model a wide range of sustainable chemical processes and optimize business benefits. Due to the nature of the complexity of the feedstocks of resid, lignin, cellulose, and plastics, it is not feasible to model them by traditional methods at a molecular level. Aspen MB AM can leverage an affordable computation burden (Table 1, O (100-300) species) to describe an upgrading process to those feedstocks while maintaining full molecular details. Thus, such an embodiment provides acceptable solution performance in industries. Moreover, MB AM allows those archipelago complexes and individual molecules to co-exist in the system and users can easily track high value individual molecules in wide range sustainable models (from complex feedstock upgrading to high valued downstream chemical or fuel unit ops such as: green diesel, sustainable aviation fuel, aromatics and ethylene). In this way, embodiments can be used to determine properties of feedstocks that could previously not be determined. Further, the results from embodiments can be used to control and modify real-world processes. For instance, an embodiment may determine the property of a feedstock subject to a manufacturing process and, in response to the determined property, modify an aspect of the manufacturing process to improve results.

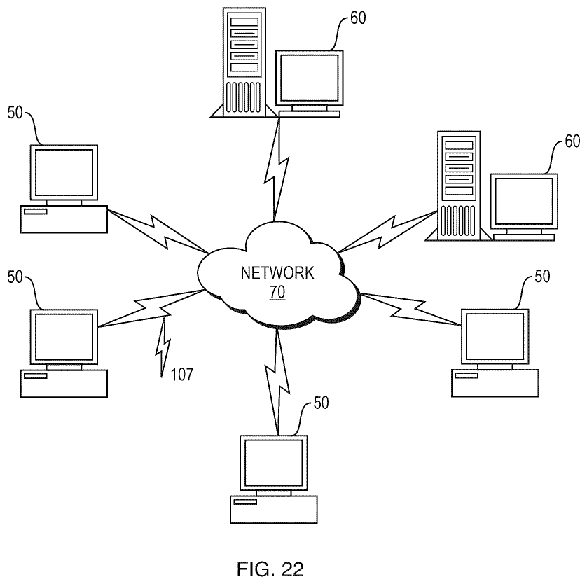

FIG. 22 illustrates a computer network or similar digital processing environment in which an embodiment may be implemented.

Client computer(s)/devices 50 and server computer(s) 60 provide processing, storage, and input/output devices executing application programs and the like. Client computer(s)/devices 50 can also be linked through communications network 70 to other computing devices, including other client devices/processes 50 and server computer(s) 60. Communications network 70 can be part of a remote access network, a global network (e.g., the Internet), cloud computing servers or service, a worldwide collection of computers, Local area or Wide area networks, and gateways that currently use respective protocols (TCP/IP, Bluetooth, etc.) to communicate with one another. Other electronic device/computer network architectures are suitable.

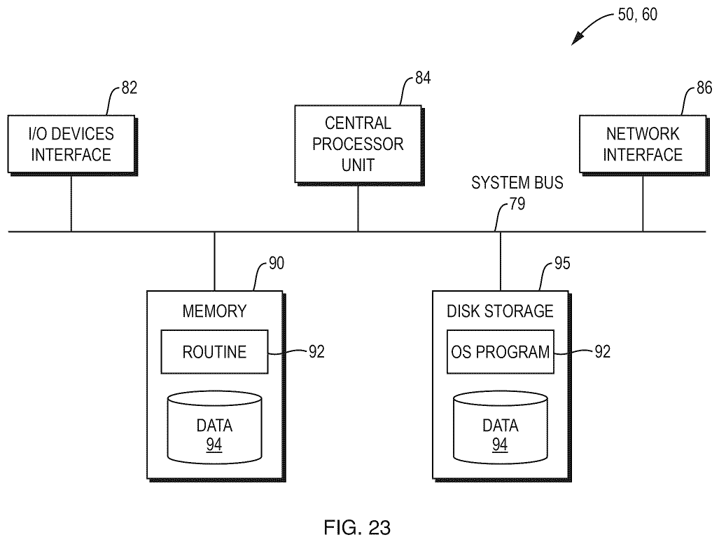

FIG. 23 is a diagram of the internal structure of a computer (e.g., client processor/device 50 or server computers 60) in the computer system of FIG. 22. Each computer 50, 60 contains system bus 79, where a bus is a set of hardware lines used for data transfer among the components of a computer or processing system. Bus 79 is essentially a shared conduit that connects different elements of a computer system (e.g., processor, disk storage, memory, input/output ports, network ports, etc.) that enables the transfer of information between the elements. Attached to system bus 79 is I/O device interface 82 for connecting various input and output devices (e.g., keyboard, mouse, displays, printers, speakers, etc.) to the computer 50, 60. Network interface 86 allows the computer to connect to various other devices attached to a network (e.g., network 70 of FIG. 22). Memory 90 provides volatile storage for computer software instructions 92 and data 94 (such as method 330, workflow 2100, MB EORXR, etc. detailed above) used to implement an embodiment of the present invention. Disk storage 95 provides non-volatile storage for computer software instructions 92 and data 94 used to implement an embodiment of the present invention. Central processor unit 84 is also attached to system bus 79 and provides for the execution of computer instructions.

In one embodiment, the processor routines 92 and data 94 are a computer program product (generally referenced 92), including a computer readable medium (e.g., a removable storage medium such as one or more DVD-ROM's, CD-ROM's, diskettes, tapes, etc.) that provides at least a portion of the software instructions for the invention system. Computer program product 92 can be installed by any suitable software installation procedure, as is well known in the art. In another embodiment, at least a portion of the software instructions may also be downloaded over a cable, communication and/or wireless connection. In other embodiments, the invention programs are a computer program propagated signal product 107 embodied on a propagated signal on a propagation medium (e.g., a radio wave, an infrared wave, a laser wave, a sound wave, or an electrical wave propagated over a global network such as the Internet, or other network(s)). Such carrier medium or signals provide at least a portion of the software instructions for the present invention routines/program 92.

In alternate embodiments, the propagated signal is an analog carrier wave or digital signal carried on the propagated medium. For example, the propagated signal may be a digitized signal propagated over a global network (e.g., the Internet), a telecommunications network, or other network. In one embodiment, the propagated signal is a signal that is transmitted over the propagation medium over a period of time, such as the instructions for a software application sent in packets over a network over a period of milliseconds, seconds, minutes, or longer. In another embodiment, the computer readable medium of computer program product 92 is a propagation medium that the computer system 50 may receive and read, such as by receiving the propagation medium and identifying a propagated signal embodied in the propagation medium, as described above for computer program propagated signal product.

Generally speaking, the term “carrier medium” or transient carrier encompasses the foregoing transient signals, propagated signals, propagated medium, storage medium and the like.

In other embodiments, the program product 92 may be implemented as a so-called Software as a Service (SaaS), or other installation or communication supporting end-users.

Embodiments or aspects thereof may be implemented in the form of hardware, firmware, or software. If implemented in software, the software may be stored on any non-transient computer readable medium that is configured to enable a processor to load the software or subsets of instructions thereof. The processor then executes the instructions and is configured to operate or cause an apparatus to operate in a manner as described herein.

Further, firmware, software, routines, or instructions may be described herein as performing certain actions and/or functions of the data processors. However, it should be appreciated that such descriptions contained herein are merely for convenience and that such actions in fact result from computing devices, processors, controllers, or other devices executing the firmware, software, routines, instructions, etc.

It should be understood that the flow diagrams, block diagrams, and network diagrams may include more or fewer elements, be arranged differently, or be represented differently. But it further should be understood that certain implementations may dictate the block and network diagrams and the number of block and network diagrams illustrating the execution of the embodiments be implemented in a particular way.

Accordingly, further embodiments may also be implemented in a variety of computer architectures, physical, virtual, cloud computers, and/or some combination thereof, and thus, the data processors described herein are intended for purposes of illustration only and not as a limitation of the embodiments.

While example embodiments have been particularly shown and described, it will be understood by those skilled in the art that various changes in form and details may be made therein without departing from the scope of the embodiments encompassed by the appended claims.

What is claimed is:

1. A computer-implemented method for determining properties of a chemical feedstock, the method comprising:
in computer memory, representing a composition of molecules in a chemical feedstock as a probabilistic combination of (i) a subset of a plurality of structural attribute representations, wherein the plurality of structural attribute representations includes cores, inter-core linkages, and free-terminal inter-core linkages and (ii) a subset of a plurality of individual molecule representations, the composition of molecules including any of an amorphous solid and a polymeric material;
determining, in the computer memory, representations of reaction paths and reaction kinetics in terms of the plurality of structural attribute representations and the plurality of individual molecule representations;
performing a simulation of a chemical process on the chemical feedstock that results in a processed feedstock, wherein the simulation is performed using (i) the subset of the plurality of structural attribute representations, (ii) the subset of the plurality of individual molecule representations, and (iii) the determined representations of the reaction paths and reaction kinetics; and
based on results of performing the simulation, determining a property of the processed feedstock.

2. The method of claim 1, wherein a given free-terminal inter-core linkage represents a side chain.

3. The method of claim 1, further comprising:
receiving, in the computer memory, an indication of at least one of: the subset of the plurality of structural attribute representations and the subset of the plurality of individual molecule representations.

4. The method of claim 1, wherein the chemical process is a chemical reaction.

5. The method of claim 4, wherein the chemical reaction is a combination reaction, a decomposition reaction, a single-replacement reaction, a double-replacement reaction, or a reactor reaction, further comprising: polymerization, depolymerization, saturation, desaturation, hydrodesulfurization (HDS), hydrodenitrogenation (HDN), ring-opening, ring-closing, addition reaction, elimination reaction, hydrodemetallization (HDM), hydrodeoxygenation (HDO), carbonylation, decarbonylation, carboxylation, decarboxylation, hydrolysis, hydroisomerization, acid cracking, metal cracking, ring condensation, or ring expansion.

6. The method of claim 1, wherein the chemical process is a separation.

7. The method of claim 6, wherein the separation is at least one of: a vapor-liquid equilibrium separation, a liquid-liquid equilibrium separation, and a solid-liquid equilibrium separation.

8. The method of claim 1, further comprising:
performing the simulation of the chemical process further using user input.

9. The method of claim 8, wherein the user input is at least one of: a physical property, a thermodynamic property, or one or more chemical structures of an attribute of the chemical feedstock.

10. The method of claim 1, wherein the chemical feedstock is at least one of: petroleum resid, coal, lignin, cellulose, and a plastic.

11. The method of claim 1, wherein the property determined for the processed feedstock is at least one of: a thermodynamic property, a physical property, and a molecular distribution.

12. A system for determining properties of a chemical feedstock, the system comprising:
a processor; and
a memory with computer code instructions stored thereon, the processor and the memory, with the computer code instructions being configured to cause the system to:
in the memory, represent a composition of molecules in a chemical feedstock as a probabilistic combination of (i) a subset of a plurality of structural attribute representations, wherein the plurality of structural attribute representations includes cores, inter-core linkages, and free-terminal inter-core linkages and (ii) a subset of a plurality of individual molecule representations, the composition of molecules including any of an amorphous solid and a polymeric material;
determine, in the memory, representations of reaction paths and reaction kinetics in terms of the plurality of structural attribute representations and the plurality of individual molecule representations;
perform a simulation of a chemical process on the chemical feedstock that results in a processed feedstock, wherein the simulation is performed using (i) the subset of the plurality of structural attribute representations, (ii) the subset of the plurality of individual molecule representations, and (iii) the determined representations of the reaction paths and reaction kinetics; and
based on results of performing the simulation, determine a property of the processed feedstock.

13. The system of claim 12, wherein a given free-terminal inter-core linkage represents a side chain.

14. The system of claim 12, wherein the processor and the memory, with the computer code instructions, are further configured to cause the system to:
receive, in the memory, an indication of at least one of: the subset of the plurality of structural attribute representations and the subset of the plurality of individual molecule representations.

15. The system of claim 12, wherein the chemical process is a chemical reaction or a separation.

16. The system of claim 12, wherein the chemical process is a chemical reaction and the chemical reaction is a combination reaction, a decomposition reaction, a single-replacement reaction, a double-replacement reaction, or a reactor reaction, further comprising: polymerization, depolymerization, saturation, desaturation, hydrodesulfurization (HDS), hydrodenitrogenation (HDN), ring-opening, ring-closing, addition reaction, elimination reaction, hydrodemetallization (HDM), hydrodeoxygenation (HDO), carbonylation, decarbonylation, carboxylation, decarboxylation, hydrolysis, hydroisomerization, acid cracking, metal cracking, ring condensation, or ring expansion.

17. The system of claim 12, wherein the chemical process is a separation and the separation is at least one of: a vapor-liquid equilibrium separation, a liquid-liquid equilibrium separation, and a solid-liquid equilibrium separation.

18. The system of claim 12, wherein the chemical feedstock is at least one of: petroleum resid, coal, lignin, cellulose, and a plastic.

19. The system of claim 12, wherein the property determined for the processed feedstock is at least one of: a thermodynamic property, a physical property, and a molecular distribution.

20. A non-transitory computer program product for determining properties of a chemical feedstock, the computer program product comprising a computer-readable medium with computer code instructions stored thereon, the computer code instructions being configured, when executed by a processor, to cause an apparatus associated with the processor to:
in computer memory, represent a composition of molecules in a chemical feedstock as a probabilistic combination of (i) a subset of a plurality of structural attribute representations, wherein the plurality of structural attribute representations includes cores, inter-core linkages, and free-terminal inter-core linkages and (ii) a subset of a plurality of individual molecule representations, the composition of molecules including any of an amorphous solid and a polymeric material;
determine, in the computer memory, representations of reaction paths and reaction kinetics in terms of the plurality of structural attribute representations and the plurality of individual molecule representations;
perform a simulation of a chemical process on the chemical feedstock that results in a processed feedstock, wherein the simulation is performed using (i) the subset of the plurality of structural attribute representations, (ii) the subset of the plurality of individual molecule representations, and (iii) the determined representations of the reaction paths and reaction kinetics; and
based on results of performing the simulation, determine a property of the processed feedstock.
